(19) United States
(12) Patent Application Publication
(10) Pub. No.: US 2022/0242929 A1
(43) Pub. Date: Aug. 4, 2022

(54) IMMUNE CELLS EXPRESSING MODIFIED CELL RECEPTORS AND METHODS OF MAKING

(71) Applicant: CARTHERICS PTY. LTD., Carlton, Victoria (AU)

(72) Inventors: Runzhe SHU, Carlton, Victoria (AU); Alan TROUNSON, Carlton, Victoria (AU); Ian NISBET, Carlton, Victoria (AU); Nicholas BOYD, Carlton, Victoria (AU); Richard BOYD, Carlton, Victoria (AU); Vera EVTIMOV, Carlton, Victoria (AU)

(73) Assignee: CARTHERICS PTY. LTD., Carlton, Victoria (AU)

(21) Appl. No.: 17/629,445

(22) PCT Filed: Aug. 4, 2020
(86) PCT No.: PCT/AU2020/050800

§ 371 Date: Jan. 24, 2022

Related U.S. Application Data
(60) Provisional application No. 62/882,674, filed on Aug. 5, 2019.

(51) Int. Cl. C07K 14/725; C07K 16/30; C07K 16/28

(52) U.S. Cl. CPC C07K 14/7051; C07K 16/3092; C07K 16/2878; C07K 16/2887; C07K 16/28; C07K 16/2803

(57) ABSTRACT

This disclosure relates to immune cells (such as T cells or NK cells) modified in their cell surface receptors to recognize one or more target antigens, in particular tumor-associated antigens. This disclosure also relates to a simple method for editing cell receptors, in particular cell surface receptors naturally expressed by immune cells such as T cells or NK cells, to create modified immune cells (e.g., cytotoxic cells) targeted against one or more target antigens, in particular tumor-associated antigens. Further, this disclosure relates to stem cells modified in one or more endogenous genes encoding one or more cell surface receptors and capable of differentiating into immune cells expressing modified cell surface receptors that recognize one or more target antigens. In addition, this disclosure relates to methods of making such modified stem cells.

### CROSS REFERENCE TO RELATED APPLICATION

This application claims the benefit of priority from U.S. Provisional Application No. 62/882,674, filed Aug. 5, 2019, the entire contents of which are incorporated herein by reference.

### INCORPORATION BY REFERENCE OF SEQUENCE LISTING

The sequence listing in the ASCII text file, named as 37440WO_SequenceListing.txt of 83KB, created on Jul. 28, 2020, submitted herewith, is incorporated herein by reference.

### FIELD OF THE DISCLOSURE

This disclosure relates to immune cells (such as T cells or NK cells) modified in their cell surface receptors to recognize one or more target antigens, in particular tumor-associated antigens. This disclosure also relates to a simple method for editing cell receptors, in particular cell surface receptors naturally expressed by immune cells such as T cells or NK cells, to create modified immune cells (e.g., cytotoxic cells) targeted against one or more target antigens, in particular tumor-associated antigens. Further, this disclosure relates to stem cells modified in one or more endogenous genes encoding one or more cell surface receptors and capable of differentiating into immune cells expressing modified cell surface receptors that recognize one or more target antigens. In addition, this disclosure relates to methods of making such modified stem cells.

### BACKGROUND

T cells expressing chimeric antigen receptors (CAR-T cells) have been shown to be very effective in killing tumor cells in diseases such as acute lymphocytic leukemia (ALL) and non-Hodgkin's lymphoma (NHL). Approved products targeting the B cell antigen CD19 are produced by introducing a CAR gene construct into patient-derived (“autologous”) T cells. Additional autologous products are in development targeting other blood cell markers such as B cell maturation antigen (BCMA) for other hematological malignancies, such as multiple myeloma.

While the clinical results with CAR-T cells in blood-based cancers have been impressive, similar results have not been forthcoming in the treatment of solid tumors. There are multiple reasons for the relative lack of efficacy in solid tumors, including restricted access to the tumor site, the immunosuppressive nature of the tumor microenvironment and the lack of solid tumor-specific target antigens. In addition, lack of persistence and “exhaustion” of the administered CAR-T cells is a consistently observed limitation.

It has been proposed that T cell exhaustion may be linked to the non-natural antigenic stimulation of T cells. Natural activation of T cells occurs via the T cell receptor (TCR) and is a highly regulated and dynamic process. In contrast, CAR-T cells incorporate a viral-engineered CAR, where strong but variegated CAR expression is driven by constitutive promoters like EF1α, CMV and PGK. While CAR constructs incorporate some TCR signalling and activation components, the process is not regulated in any way other than the “on-off” switch of the CAR binding to its target. The natural TCR is a complex of six distinct receptor subunits, which recognise foreign antigens in the context of presentation by class I HLA molecules. In contrast, CARs utilise the signalling domain of just one TCR subunit, artificially linked to a co-stimulatory domain from a different receptor. It has been proposed that this non-natural activation results in over-stimulation of the CAR-T cells, leading to exhaustion and loss of function.

To try and address the T cell exhaustion problem, a new type of artificial T cell receptor has been reported that combines the target flexibility of a CAR with the regulated activation of the core TCR structure. This T cell engineering platform has been designated as T cell receptor fusion proteins (TFPs). To create TFPs, a DNA construct was generated that contained the coding sequence for an antibody single-chain variable fragment (scFv) at the N-terminal end of a T cell receptor subunit gene, and then introducing the DNA construct into a T cell using a viral vector. The TFP gene expression cassettes inserted into the T cell genome, resulting in T cells expressing (under a constitutive promoter) the TFP in addition to the normal TCR subunit. The over-expressed TFP proteins competed with the natural TCR subunits for incorporation into the TCR complex thereby, at some frequency, generating T cells with the ability to recognize and kill cells expressing the target specified by the scFv.

While pre-clinical results with TFP cells are promising, the manufacture of TFP cells using the reported methods is cumbersome, imprecise and inefficient and, therefore, not well suited to a commercial product. CAR-T cells are typically produced through transduction of γ-retroviral or lentiviral vectors carrying the CAR constructs into the T cells. This is also the basic method used for the generation of the reported TFP cells. However, retroviral or lentiviral transduction results in random integration of the CAR (or TFP) genes into the genome, which creates a number of disadvantages and risks in the quality of the resulting product, including: variation in transgene expression; functional gene silencing; potential oncogenic transformation and associated clonal expansion of oncogenic T cells. In addition, because of random gene insertion, the final product is a heterogenous mixture of different T cell clones, each of which will have a different expression pattern for the encoded TFP.

Moreover, the requirement for cGMP grade viral vectors has significant time and cost implications for the development and manufacturing cost of CAR-T cells. T cell products based on the TTPs reported will suffer the same manufacturing cost and time challenges as have been observed with existing CAR-T cell products. Recent studies using CRISPR-Cas9, TALEN or ZFN technologies have been shown to deliver CAR transgenes efficiently into specific loci, including safe harbour sites like AAVS1, ROSA26 or TRAC. The resultant CAR-T cells showed uniform CAR expression and site-specific integration, However, to achieve an acceptable transgene expression efficiency (more than 20%), these methods still require a viral delivery system [in this case adeno-associated virus (AAV)] to deliver the CAR gene as the donor DNA into T cells. Though several forms of donor DNA, such as double-stranded DNA, single-stranded DNA or plasmid DNA could be delivered into T cells using electroporation or nucleofection, the efficiency of delivering CAR, TFP or TCR gene expression cassettes into T cells was very low (less than 15%). The invention described in this disclosure overcomes these limitations, as it can achieve around 20-40% of transgene expression in T cells without using viral vectors. Moreover, the T cells disclosed herein can be expanded in vitro for immunotherapy.

In addition, the current invention extends the TTP concept beyond the modification of TCR subunits. It provides a simple, efficient method for converting any cell surface receptor that triggers a “killing” function into a targeting receptor, thereby opening up the possibility of developing an extensive range of targeting killer cells expressing different receptors activating different killing mechanisms.

### SUMMARY OF THE DISCLOSURE

In one aspect, this disclosure provides a method for generating a modified immune cell that recognizes a target antigen. The method comprises inserting a nucleic acid sequence encoding an antigen recognition moiety for the target antigen into an endogenous cell receptor gene in an immune cell to form a modified cell receptor gene, thereby generating a modified immune cell comprising the modified cell receptor gene, wherein the insertion is at a specific site in the coding region of the endogenous cell receptor gene such that the endogenous cell receptor is modified to include the antigen recognition moiety in the extracellular domain, and wherein expression of the modified cell receptor gene is under control of the endogenous cis-regulatory elements at the endogenous cell receptor gene locus.

In some embodiments, the nucleic acid sequence is introduced by a non-viral delivery method.

In some embodiments, the insertion is an in-frame insertion within the extracellular domain-coding sequence in an endogenous cell receptor gene. In some embodiments, the insertion is within 5 codons from the codon encoding the free end amino acid of the extracellular domain of the endogenous cell receptor. In some embodiments, the insertion is an in-frame addition immediately following or followed by the codon encoding the free end amino acid of the extracellular domain of an endogenous cell receptor (such that the antigen recognition moiety is added at the free end of the extracellular domain of the endogenous cell receptor).

In some embodiments, the modified immune cell expresses a modified cell receptor, and does not express the unmodified endogenous cell receptor.

In some embodiments, two or more modified cell receptor genes are generated to create immune cells that recognize two or more different target antigens. In some embodiments, two of CD3ϵ, CD3γ and CD3δ receptor genes in T cells have been modified to create modified T cells that recognize two target antigens, e.g., two target antigens selected from TAG-72, CD19, CD20, CD47, folate receptor alpha (FRa), or BCMA. In some embodiments, one or more of CD3ϵ, CD 3γ and CD3δ receptor genes and at least one non-CD3 receptor gene (e.g., CD28) in T cells have been modified to create modified T cells that recognize two or more target antigens, e.g., two or more target antigens selected from TAG-72, CD19, CD20, CD47, folate receptor alpha (FRa), or BCMA.

In some embodiments, the immune cell that has been modified is selected from a T cell, a NKT cell or a NK cell.

In some embodiments, the cell receptor is an interleukin receptor,

In some embodiments, the cell receptor being modified is selected from, CD2,CD3, CD4, CD8, TCR, CD28, NKG2D, CD94, CD16, NKp46, NKp30, NKp44, 2B4, killer-cell immunoglobulin-like receptor (KIR) CD69, CD27, 2B4, DNAM1, DAP10, DAP12, FcRγ, IL-2Rα, IL-12R, IL-15Rα, IL-18R, or IL-21R. In some embodiments, the cell receptor is selected from CD3ϵ, CD3γ or CD3δ. In some embodiments, the cell receptor is selected from TCRα or TCRβ or TCRγ or TCRδ.

In some embodiments, the target antigen is a cell surface protein. In some embodiments, the target antigen is a tumor associated antigen. In some embodiments, the target antigen is selected from TAG-72, CD19, CD20, CD47, folate receptor alpha (FRa), or BCMA. In some embodiments, the target antigen is a viral protein or a cell surface receptor or co-receptor that serves as the site for viral binding to a cell.

In some embodiments, the antigen recognition moiety for a targeted antigen comprises an antibody fragment. In some embodiments, the antigen recognition moiety for a targeted antigen is a scFv.

In some embodiments, the nucleic acid sequence inserted encodes an antigen recognition moiety and a linker. In some embodiments, the size of the nucleic acid sequence being inserted is less than 1.5 kb.

In some embodiments, the insertion at a specific site is directed by a CRISPR/Cas9, TALEN or ZEN system. In some embodiments, the insertion is directed by CRISPR/Cas9 by using a Cas9 nuclease and a guide RNA. Examples of guide RNAs include those listed in Table 1, e.g., SEQ ID NO: 1, SEQ ID NO: 2, SEQ ID NO: 3, SEQ ID NO: 4, SEQ ID NO: 5 and SEQ ID NO: 6.

In another aspect, a method is provided for generating modified immune cells that recognize a target antigen, comprising (i) introducing to a population of immune cells, a nucleic acid sequence encoding an antigen recognition moiety for the target antigen, for insertion into an endogenous cell receptor gene in the immune cells to form a modified cell receptor gene, wherein the insertion is at a specific site in the coding region of the endogenous cell receptor gene such that the endogenous cell receptor is modified to include the antigen recognition moiety in the extracellular domain, and wherein expression of the modified cell receptor gene is under control of the endogenous cis-regulatory elements at the endogenous cell receptor gene locus; and (ii) obtaining a cell population, wherein at least a. portion of the cells in the cell population are modified immune cells that comprise the modified cell receptor gene. In some embodiments, at least 5%, 10%, 15%, 20%, 25%, or 30% of the cells in the cell population are modified immune cells. In some embodiments, at least 20%, or greater (e.g., 21%, 22%, 23%, 24%, 25%, 26%, 27%, 28%, 29%, 30%, or greater) of the cells in the cell population are modified immune cells.

In some embodiments, the method further comprises isolating the modified immune cells that comprise a modified cell receptor gene.

In some embodiments, two or more nucleic acid sequences are introduced into immune cells, either sequentially or simultaneously, to modify two or more cell receptor genes; and a cell population is obtained, wherein at least a portion of the cells in the cell population are modified immune cells that comprise the two or more modified cell receptor genes and recognize two or more target antigens. In some embodiments, at least two of CD3ϵ, CD3γ and CD3δ receptor genes in T cells have been modified to create modified T cells that recognize two or more target antigens, e.g., two or more target antigens selected from TAG-72, CD19, CD20, CD47, folate receptor alpha (FRa), or BCMA. In some embodiments, one or more of CD3ϵ, CD3γ and CD3δ receptor genes and at least one non-CD3 receptor gene (e.g., CD28) in T cells have been modified to create modified T cells that recognize two or more target antigens, e.g., two or more target antigens selected from TAG-72, CD19, CD20, CD47, folate receptor alpha (FRa), or BCMA.

In another aspect, this disclosure provides a method for generating a modified stem cell capable of differentiating into an immune cell that recognizes a target antigen. The method comprises inserting a nucleic acid sequence encoding an antigen recognition moiety for the target antigen into an endogenous gene in a stem cell encoding a cell receptor, thereby generating a modified stem cell comprising a modified cell receptor gene and capable of differentiating into an immune cell comprising the modified cell receptor gene and expressing the modified cell receptor. The insertion is at a specific site in the coding region of the endogenous cell receptor gene such that the cell receptor expressed on the immune cell differentiated from the modified stem cell includes the antigen recognition moiety in the extracellular domain, and wherein expression of the modified cell receptor gene is under control of the endogenous cis-regulatory elements at the endogenous cell receptor gene locus.

In some embodiments, the nucleic acid sequence is introduced by a non-viral delivery method.

In some embodiments, the insertion is an in-frame insertion within the extracellular domain-coding sequence in an endogenous gene encoding a cell receptor. In some embodiments, the insertion is within 5 codons from the codon encoding the free end amino acid of the extracellular domain of the cell receptor. In some embodiments, the insertion is an in-frame addition immediately following or followed by the codon encoding the free end amino acid of the extracellular domain of a cell receptor (such that the antigen recognition moiety is added at the free end of the extracellular domain of the cell receptor).

In some embodiments, two or more modified cell receptor genes are generated to create a modified stem cell capable of differentiating into an immune cell that recognizes two or more different target antigens. In some embodiments, two or more of CD3ϵ, CD3γ and CD3δ receptor genes in stem cells have been modified to create modified stem cells capable of differentiating into T cells that recognize two or more target antigens, e.g., two or more target antigens selected from TAG-72, CD19, CD20, CD47, folate receptor alpha (FRa), or BCMA. In some embodiments, one or more of CD3ϵ, CD3γ and CD3δ receptor genes and at least one non-CD3 receptor gene (e.g., CD28) in stem cells have been modified to create modified stem cells capable of differentiating into cells that recognize two or more target antigens, e.g., two or more target antigens selected from TAG-72, CD19, CD20, CD47, folate receptor alpha (FRa), or BCMA.

In some embodiments, the immune cell which can be derived from a stem cell is selected from a T cell, a NKT cell or a NK cell.

In some embodiments, the cell receptor is an interleukin receptor.

In some embodiments, the cell receptor being modified is selected from, CD2,CD3, CD4, CD8, TCR, CD28, NKG2D, CD94, CD16, NKp46, NKp30, NKp44, 2B4, CD69, CD27, 2B4, DNAM1, DAP10, DAP12, FcRγ, IL-2Rα, IL-12R, IL-15Rα, IL-18R, or IL-21R. In some embodiments, the cell receptor is selected from CD3ϵ, CD3γ or CD3δ. In some embodiments, the cell receptor is selected from TCRα, TCRβ, TCRγ, or TCRδ.

In some embodiments, the target antigen is a cell surface protein. In some embodiments, the target antigen is a tumor associated antigen. In some embodiments, the target antigen is selected from TAG-72, CD19, CD20, CD47, folate receptor alpha (FRa), or BCMA. In some embodiments, the target antigen is a viral protein, or a cell surface receptor or co-receptor that serves as the site for viral binding to a cell.

In some embodiments, the antigen recognition moiety for a targeted antigen comprises an antibody fragment. In some embodiments, the antigen recognition moiety for a targeted antigen is a scFv.

In some embodiments, the nucleic acid sequence inserted encodes an antigen recognition moiety and a linker. In some embodiments, the size of the nucleic acid sequence being inserted is less than 1.5 kb.

In some embodiments, the insertion at a specific site is directed by a CRISPR/Cas9, TALEN or ZEN system. In some embodiments, the insertion is directed by CRISPR/Cas9 by using a Cas9 nuclease and a guide RNA. Examples of guide RNAs include those listed in Table 1, e.g., SEQ ID NO: 1-SEQ ID NO: 6, SEQ ID NO: 26-SEQ ID NO: 94.

In some embodiments, the method further comprises culturing a modified stem cell generated to differentiate the modified stem cell into an immune cell, e.g., T cell, a NKT cell or a NK cell.

In another aspect, a method is provided for generating modified stem cells capable of differentiating into immune cells that recognize a target antigen, comprising (i) introducing to a population of stem cells, a nucleic acid sequence encoding an antigen recognition moiety for the target antigen, for insertion into an endogenous gene in the stem cells encoding a cell receptor, to form a modified cell receptor gene, wherein the insertion is at a specific site in the coding region of the endogenous cell receptor gene such that the cell receptor expressed on the immune cell includes the antigen recognition moiety in the extracellular domain, and wherein expression of the modified cell receptor gene is under control of the endogenous cis-regulatory elements at the endogenous cell receptor gene locus; and (ii) obtaining a cell population, wherein at least a portion of the cells in the cell population are modified stem cells that comprise the modified cell receptor gene. In some embodiments, at least 5%, 10%, 15%, 20%, 25%, or 30% of the cells in the cell population are modified stem cells. In some embodiments, at least 20%, or greater (e.g., 21%, 22%, 23%, 24%, 25%, 26%, 27%, 28%, 29%, 30%, or greater) of the cells in the cell population are modified stem cells.

In some embodiments, the method further comprises isolating the modified stem cells that comprise a modified cell receptor gene.

In some embodiments, two or more nucleic acid sequences are introduced into stem cells, either sequentially or simultaneously, to modify two or more cell receptor genes; and a cell population is obtained, wherein at least a portion of the cells in the cell population are modified stem cells that comprise the two or more modified cell receptor genes encoding modified cell receptors that recognize two or more target antigens. In some embodiments, two or more of CD3ϵ, CD3γ and CD3δ receptor genes in stem cells have been modified to create modified stem cells capable of differentiating into T cells that recognize two or more target antigens, e.g., two or more target antigens selected from TAG-72, CD19, CD20, CD47, folate receptor alpha (FRa), or BCMA. In some embodiments, one or more of CD3ϵ, CD3γ and CD3δ receptor genes and at least one non-CD3 receptor gene (e.g., CD28) in stem cells have been modified to create modified stem cells capable of differentiating into T cells that recognize two or more target antigens, e.g., two or more target antigens selected from TAG-72, CD19, CD20, CD47, folate receptor alpha (FRa), or BCMA.

In some embodiments, the method further comprises culturing a cell population comprising modified stem cells to differentiate the modified stem cells into immune cells, e.g., T cell, a NKT cell or a NK cell.

In a further aspect, provided herein is a modified immune cell produced by the methods disclosed herein. In some embodiments, the modified immune cell is cytotoxic against cells expressing the target antigen.

In still a further aspect, provided herein is a cell population comprising modified immune cells produced by the methods disclosed herein. In some embodiments, the modified immune cells in the cell population are cytotoxic against cells expressing the target antigen.

In one aspect, a modified immune cell is provided that recognizes a target antigen, and comprises in its genome, a nucleic acid sequence encoding an antigen recognition moiety for the target antigen, inserted in an endogenous cell receptor gene to form a modified cell receptor gene, wherein the insertion is at a specific site in the coding region of the endogenous cell receptor gene such that the endogenous cell receptor is modified to include the antigen recognition moiety in the extracellular domain, and wherein expression of the modified cell receptor gene is under control of the endogenous cis-regulatory elements at the endogenous cell receptor gene locus.

In some embodiments, the insertion is an in-frame insertion within the extracellular domain-coding sequence in an endogenous cell receptor gene. In some embodiments, the insertion is within 5 codons from the codon encoding the free end amino acid of the extracellular domain of the endogenous cell receptor. In some embodiments, the insertion is an in-frame insertion immediately following or followed by the codon encoding the free end amino acid of the extracellular domain of an endogenous cell receptor (such that the antigen recognition moiety is added at the free end of the extracellular domain of the endogenous cell receptor).

In some embodiments, the modified immune cell comprises two or more modified cell receptor genes and recognizes two or more different target antigens. In some embodiments, two or more of CD3ϵ, CD3γ and CD3δ receptor genes in T cells have been modified to create modified T cells that recognize two or more target antigens, e.g., two or more target antigens selected from TAG-72, CD19, CD20, CD47, folate receptor alpha (FRa), or BCMA. In some embodiments, one or more of CD3ϵ, CD3γ and CD3δ receptor genes and at least one non-CD3 receptor gene (e.g., CD28) T cells have been modified to create modified T cells that recognize two or more target antigens, e.g., two or more target antigens selected from TAG-72, CD19, CD20, CD47, folate receptor alpha (FRa), or BCMA

In some embodiments, the modified immune cell expresses a modified cell receptor(s), and does not express the unmodified endogenous cell receptor(s).

In some embodiments, the immune cell is selected from a T cell, an NKT cell or an NK cell.

In some embodiments, the cell receptor is an interleukin receptor.

In some embodiments, the cell receptor is selected from CD2, CD3, CD4, CD8, TCR, CD28, NKG2D, CD94, CD16, NKp46, NKp30, NKp44, 2B4, CD69, CD27, 2B4, DNAM1, DAP10, DAP12, FcRγ, IL-2Rα, IL-12R, IL-15Rα, IL-18R or IL-21R. In some embodiments, the cell receptor is selected from CD3ϵ, CD3γ or CD3δ. In some embodiments, the cell receptor is selected from TCRα, TCRβ, TCRγ, TCRδ.

In some embodiments, the target antigen is a cell surface protein. In some embodiments, the target antigen is a tumor associated antigen. In some embodiments, the target antigen is selected from TAG-72, CD19, CD20, CD47, folate receptor alpha (FRa), or BCMA. In some embodiments, the target antigen is a viral protein, or a cell surface receptor or co-receptor that serves as the site for viral binding to a cell.

In some embodiments, the antigen recognition moiety for a targeted antigen comprises an antibody fragment. In some embodiments, the antigen recognition moiety for a targeted antigen is a scFv.

In some embodiments, the nucleic acid sequence inserted encodes an antigen recognition moiety and a linker.

In some embodiments, the size of the nucleic acid sequence inserted is less than 1.5 kb.

In one aspect, a cell population is provided that comprises modified immune cells that recognize a target antigen, wherein the modified immune cells each comprise in the genome a nucleic acid sequence encoding an antigen recognition moiety for the target antigen inserted in an endogenous cell receptor gene to form a modified cell receptor gene, wherein the insertion is at a specific site in the coding region of the endogenous cell receptor gene such that the endogenous cell receptor is modified to include the antigen recognition moiety in the extracellular domain, and wherein expression of the modified cell receptor gene is under control of the endogenous cis-regulatory elements at the endogenous cell receptor gene locus.

In some embodiments, the modified immune cells each comprise in the genome two or more modified cell receptor genes and recognize two or more different target antigens. In some embodiments, two or more of CD3ϵ, CD3γ and CD3δ receptor genes in T cells have been modified to create modified T cells that recognize two or more target antigens, e.g., two or more target antigens selected from TAG-72, CD19, CD20, CD47, folate receptor alpha (FRa), or BCMA. In some embodiments, one or more of CD3ϵ, CD3γ and CD3δ receptor genes and at least one non-CD3 receptor gene (e.g., CD28) in stem cells have been modified to create modified stem cells capable of differentiating into T cells that recognize two or more target antigens, e.g., two or more target antigens selected from TAG-72, CD19, CD20, CD47, folate receptor alpha (FRa), or BCMA.

In another aspect, provided herein is a pharmaceutical composition comprising a modified immune cell disclosed herein.

In still another aspect, provided herein is pharmaceutical composition comprising a cell population of modified immune cells disclosed herein.

In one aspect, a modified stem cell is provided that comprises in its genome, a nucleic acid sequence encoding an antigen recognition moiety for a target antigen, inserted in an endogenous gene encoding a cell receptor to form a modified cell receptor gene, wherein the insertion is at a specific site in the coding region of the endogenous cell receptor gene such that the cell receptor is modified to include the antigen recognition moiety in the extracellular domain, wherein expression of the modified cell receptor gene is under control of the endogenous cis-regulatory elements at the endogenous cell receptor gene locus, and wherein the modified stem cell is capable of differentiating into an immune cell that expresses the modified cell receptor which recognizes the target antigen.

In some embodiments, the insertion is an in-frame insertion within the extracellular domain-coding sequence in an endogenous gene encoding a cell receptor. In some embodiments, the insertion is within 5 codons from the codon encoding the free end amino acid of the extracellular domain of the cell receptor. In some embodiments, the insertion is an in-frame insertion immediately following or followed by the codon encoding the free end amino acid of the extracellular domain of a cell receptor (such that the antigen recognition moiety is added at the free end of the extracellular domain of the cell receptor).

In some embodiments, the modified stem cell comprises two or more modified cell receptor genes and recognizes two or more different target antigens. In some embodiments, two or more of CD3ϵ, CD3γ and CD3δ receptor genes in stem cells have been modified to create modified stem cells capable of differentiating into T cells that recognize two or more target antigens, e.g., two or more target antigens selected from TAG-72, CD19, CD20, CD47, folate receptor alpha (FRa), or BCMA. In some embodiments, one or more of CD3ϵ, CD3γ and CD3δ receptor genes and at least one non-CD3 receptor gene (e.g., CD28) in stem cells have been modified to create modified stem cells capable of differentiating into T cells that recognize two or more target antigens, e.g., two or more target antigens selected from TAG-72, CD19, CD20, CD47, folate receptor alpha (FRa), or BCMA.

In some embodiments, the modified stem cell is capable of differentiating into an immune cell selected from a cell, an NKT cell or an NK cell,

In some embodiments, the cell receptor is an interleukin receptor.

In some embodiments, the cell receptor is selected from CD2, CD3, CD4, CD8, TCR, CD28, NKG2D, CD94, CD16, NKp46, NKp30, NKp44, 2B4, CD69, CD27, 2B4, DNAM1, DAP10, DAP12, FcRγ, IL-2Rα, IL-12R, IL-15Rα, IL-18R or IL-21R. In some embodiments, the cell receptor is selected from CD3ϵ, CD3γ or CD3δ. In some embodiments, the cell receptor is selected from TCRα, TCRβ, TCRγ, or TCRδ.

In some embodiments, the target antigen is a cell surface protein, In some embodiments, the target antigen is a tumor associated antigen. In some embodiments, the target antigen is selected from TAG-72, CD19, CD20, CD47, or folate receptor alpha (FRa) or BCMA. In some embodiments, the target antigen is a viral protein, or a cell surface receptor or co-receptor that serves as the site for viral binding to a cell.

In some embodiments, the antigen recognition moiety for a targeted antigen comprises an antibody fragment. In some embodiments, the antigen recognition moiety for a targeted antigen is a scFv.

In some embodiments, the nucleic acid sequence inserted encodes an antigen recognition moiety and a linker.

In some embodiments, the size of the nucleic acid sequence inserted is less than 1.5 kb.

In one aspect, a cell population is provided that comprises modified stem cells each comprising in the genome a nucleic acid sequence encoding an antigen recognition moiety for a target antigen inserted in an endogenous gene encoding a cell receptor to form a modified cell receptor gene, wherein the insertion is at a specific site in the coding region of the endogenous cell receptor gene such that the cell receptor is modified to include the antigen recognition moiety in the extracellular domain, wherein expression of the modified cell receptor gene is under control of the endogenous cis-regulatory elements at the endogenous cell receptor gene locus, and wherein the modified stem cells are capable of differentiating into immune cells that express the modified cell receptor which recognizes the target antigen.

In some embodiments, the modified stem cells each comprise in the genome two or more modified cell receptor genes encoding two or more modified cell receptors that recognize two or more different target antigens. In some embodiments, two or more of CD3ϵ, CD3γ and CD:3δreceptor genes have been modified to create modified stem cells capable of differentiating into T cells that recognize two or more target antigens, e.g., two target antigens selected from TAG-72, CD19, CD20, CD47, folate receptor alpha (FRa), or BCMA. In some embodiments, one or more of CD3ϵ, CD3γ and CD3δ receptor genes and at least one non-CD3 receptor gene (e.g., CD28) in stem cells have been modified to create modified stem cells capable of differentiating into T cells that recognize two or more target antigens, e.g., two or more target antigens selected from TAG-72, CD 9, CD20, CD47, folate receptor alpha (FRa), or BCMA.

In one aspect, a nucleic acid construct is provided that comprises a nucleic acid sequence encoding an antigen recognition moiety that recognizes a target antigen, flanked by a 5′ homology arm and a 3′ homology arm, wherein the 5′ and 3′ homology arms are homologous to the nucleotide sequences upstream and downstream of a specific site in the coding region of an endogenous cell receptor gene in an immune cell or a stem cell capable of differentiating into an immune cell, and mediate insertion of the nucleic acid sequence into the site to form a modified cell receptor gene, such that the cell receptor is modified to include the antigen recognition moiety in the extracellular domain, and wherein expression of the modified cell receptor gene is under control of the endogenous cis-regulatory elements at the endogenous cell receptor gene locus.

In some embodiments, the insertion is an in-frame insertion within the extracellular domain-coding sequence in an endogenous cell receptor gene. In some embodiments, the insertion is within 5 codons from the codon encoding the free end amino acid of the extracellular domain of the cell receptor. In some embodiments, the insertion is an in-frame insertion immediately following or followed by the codon encoding the free end amino acid of the extracellular domain of a cell receptor (such that the antigen recognition moiety is added at the free end of the extracellular domain of the cell receptor).

In some embodiments, the construct does not include a viral nucleotide sequence.

In some embodiments, the immune cell is selected from a T cell, a NKT cell or a NK cell.

In some embodiments, the cell receptor is an interleukin receptor.

In some embodiments, the cell receptor is selected from CD2, CD3, CD4, CD8, TCR, CD28, NKG2D, CD94, CD16, NKp46, NKp30, NKp44, 2B4, CD69, CD27, 2B4, DNAM1, DAP10, DAP12, FcRγ, IL-2Rα, IL-12R, IL-15Rα, IL-18R or IL-21R. In some embodiments, the cell receptor is selected from CD3ϵ, CD3γ or CD3δ. In some embodiments, the cell receptor is selected from TCRα, TCRβ, TCRγ, TCRδ.

In some embodiments, the target antigen is a cell surface protein. In some embodiments, the target antigen is a tumor associated antigen. In some embodiments, the target antigen is selected from TAG-72, CD19, CD20, CD47, folate receptor alpha (FRa), or BCMA. In some embodiments, the target antigen is a viral protein, or a cell surface receptor or co-receptor that serves as the site for viral binding to a cell.

In some embodiments, the antigen recognition moiety for a targeted antigen comprises an antibody fragment. In some embodiments, the antigen recognition moiety for a targeted antigen is a scFv.

In some embodiments, the nucleic acid sequence encodes an antigen recognition moiety and a linker.

In some embodiments, the size of the nucleic acid sequence inserted is less than 1.5 kb.

In a further aspect, a kit is provided that comprises a nucleic acid construct disclosed herein and a guide RNA designed to target a Cas9 mediated insertion of the nucleic acid sequence at a specific site.

In another aspect, a kit is provided that comprises a first nucleic acid construct disclosed herein and a second nucleic acid construct disclosed herein, wherein the antigen recognition moiety encoded by the first nucleic acid construct recognizes a first target antigen, and the antigen recognition moiety encoded by the second nucleic acid construct recognizes a second target antigen, and wherein the first and second target antigens are different.

In yet another aspect, there is provided a method of treating a disease associated with expression of a target antigen, comprising administering a pharmaceutical composition comprising a cytotoxic cell generated by a method described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Strategy for knock-in of an anti-tumor CAR or GFP expression cassette into the AAVS1 (PPP1R12C gene) locus of human genome using CRISPR-Cas9 technology. Donor DNA can be double-stranded DNA or single-stranded DNA, containing two homologous arms, a constitutive promoter, a polyA terminator and the CAR or GFP open reading frame. Co-transfection of donor DNA with Cas9 ribonucleoprotein (RNP) complex introduces the CAR or GFP expression cassette into the AAVS1 locus through homology-directed repair (HDR).

FIG. 2. Flow cytometry analysis of GFP expression in T cells 7 days after co-transfection of AAVS1 guide RNA [SEQ ID NO: 7] formed Cas9 RNP and 2 ug GFP donor DNA, demonstrating the knock-in (KI) efficiency of the GFP cassette into the AAVS1 locus. Panel A: T cells transfected with AAVS1 Cas9 RNP alone. Panel B: T cells co-transfected with AAVS1 Cas9 RNP and GFP single-stranded donor DNA [SEQ ID NO: 13]. Panel C: T cells co-transfected with AAVS1 RNP and 2786bp GFP double-stranded donor DNA [SEQ:ID NO: 13]. Panel ID: T cells co-transfected with AAV RNP and 2386bp GFP double-stranded donor DNA [SEQ ID NO: 14]. Panel E: T cells co-transfected with AAVS1 RNP and 1985bp GIP double-stranded donor DNA [SEQ ID NO: 15], Panel F: T cells co-transfected with AAVS1 RNP and 1786bp GFP double-stranded donor DNA [SEQ ID NO: 16]).

FIG. 3. FACS analysis of GFP or CAR expression in T cells 8 days after co-transfection of AAVS1 guide RNA [SEQ ID NO: 7] formed Cas9 RNP and 2 ug GFP or anti-TAG-72 CAR donor DNA, demonstrating the knock-in (KI) efficiency GFP or CAR cassette into AAVS1 locus. Panels A and D. Non-transfected T cells, Panel B: T cells co-transfected with AAVS1 Cas9 RNP and GFP double-stranded donor DNA with the 1178bp EF1a promoter [SEQ ID NO: 17]. Panel C: T cells co-transfected with AAVS1 RNP and GFP double-stranded donor DNA with the 546bp EF I a promoter [SEQ ID NO: 14]. Panel E: T cells transfected with AAVS1 RNP and then transduced with rAAV6 containing the anti-TAG-72 CAR donor DNA [SEQ ID NO: 18]. Panel F: T cells co-transfected with AAVS1 RNP and anti-TAG-72 CAR double-stranded donor DNA [SEQ ID NO: 19].

FIG. 4. FACS analysis of CAR expression (Panels A to D) or TCR expression (Panels E to H) in T cells 10 days after co-transfection of TRAC guide RNA formed Cas9 RNP and 2 ug anti-TAG-72 CAR donor DNA, demonstrating the knock-in (KI) efficiency CAR cassette into TRAC locus. Panels A and E: Non-transfected T cells. Panels B and F: T cells transfected with TRAC Cas9 RNP alone. Panel C and G: T cells co-transfected with TRAC Cas9 RNP and CAR double-stranded donor DNA with the 1178bp EF1α promoter [SEQ ID NO: 20], Panels D and H: T cells co-transfected with TRAC RNP and CAR double-stranded donor DNA with the 546bp EF1α promoter [SEQ ID NO: 21].

FIGS. 5A and SB, Schematic diagrams of gene engineering strategy and constructs. A. Strategy for knock-in of anti-tumor antigen say sequence into the CD3ϵ gene locus of human genome using CRISPR-Cas9 technology to produce fusion protein (FP) T cells. B. Expanded view of donor DNA. Donor DNA can be double- or single-stranded DNA, containing two homologous arms, antibody scFv sequence and linkers. Co-transfection of donor DNA with Cas9 ribonucleoprotein (RNP) complex introduces the scFv coding sequence to the amino terminus of the CD3ϵ gene through homology-directed repair (HDR).

FIG. 6. Transfection of CD3ϵ CRISPR gRNA-1 [SEQ ID NO: 1] formed CD3ϵ Cas9 RNP introduces indels into CD3ϵ Exon 3. Frequency of insertions and deletions (Indel %) assessed by Inference of CRISPR Edits (ICE) assay was determined as 88% of genomic DNA 4 days after CD3ϵ RNP transfection. The nucleotide sequences shown at the bottom of the figure are set forth, from top to bottom, in SEQ ID NO: 97, SEQ ID NO: 98, SEQ ID NO: 99, SEQ ID NO: 100, SEQ ID NO: 101, SEQ ID NO: 102, and SEQ ID NO: 103, respectively.

FIG. 7. FACS analysis of surface expression of CD3 (Panels A to C) and TCRα/β (Panels D to F) in T cells 4 days after CD3ϵ RNPs transfection, demonstrating the knock-out (KO) efficiency of CD3ϵ RNPs. Panels A and D: Non-transfected T cells. Panels B and F: I cells transfected with CD3ϵ CRISPR gRNA-1 [SEQ ID NO: 1]. Panels C and F: T cells transfected with CD3ϵ CRISPR gRNA-2 [SEQ ID NO: 2]. Transfection with either gRNA-1 [SEQ ID NO: 1] or gRNA-2 ISM ID NO: 2 formed CD3ϵ Cas9 RNPs disrupts the CD3 and TCR receptor of human CD3+ T cells.

FIG. 8. FACS analysis of surface expression of anti-TAG-72 scFv (Flag) (Panels A, D, G and J), CD3 (Panels B, E, H and K) and TCRα/β (Panels C, F, I and L) in T cells 8 days after transfection, demonstrating the knock-in (KI) efficiency of anti-TAG-72 scFv DNA. Panels A, B and C: Non-transfected T cells. Panels D, E and F: T cells transfected with CD3ϵ RNP alone. Panels G, H and I: T cells transfected with CD3ϵ RNP and 1.55 ug anti-TAG-72 scFv donor DNA. Panels J, K and L; cells transfected with CD3ϵ RNP and 3.1 ug anti-TAG-72 scFv donor DNA.

FIG. 9. Growth curve of anti-TAG-72/CD3ϵ CRISPR FP T cells. 1×10e6 activated CD3 T cells were transfected with CD3ϵ gRNA-1 [SEQ ID NO: 1] formed Cas9 RNP and 2 ug of anti-TAG-72 scFv donor DNA [SEQ ID NO: 8] using Lonza 4D nucleofector to create the anti-TAG-72/CD3ϵ CRISPR FP T cells. To knock-in the anti-TAG-72 CAR expression cassette into AAVS1 or TRAC locus, anti-TAG-72-4-1BBzeta CAR donor DNAs with short EF1a promoter [SEQ ID NO: 19 or 21], were also transfected with Cas9 RNPs targeting AAVS1 or TRAC locus respectively using Lonza 4D nucleofector. Non-transfected T cells and lentiviral transduced anti-TAG-72 CAR T cells were used as control. All these T cells were grown in T cell expansion medium and cell numbers determined by live cell counting. Representative data of at least 3 independent experiments using T cells from different healthy donors. (NT: Non-transfected T cell control; lentiviral anti-TAG-72 CAR: CAR-T cells transduced by anti-TAG-72-4-1BBzeta lentiviral vector; CRP CD3e-TAG-72: T cells transfected with CD3ϵ RNP and 2 ug anti-TAG-72 scFv donor DNA; CRP AAVS1-EF1a(Short)-TAG-72 CAR: T cells transfected with AAVS1 RNP and 2 ug anti-TAG-72-4-1BBzeta CAR with short EF1a promoter donor DNA; CRP TRAC-EF1a(Short)-TAG-72 CAR: T cells transfected with TRAC RNP and 2 ug anti-TAG-72-4-1BBzeta CAR with short EF1a promoter donor DNA).

FIGS. 10A and 10B. Anti-TAG-72/CD3ϵ CRISPR FP T cells mediate potent cell killing of TAG-72hi expressing target cells (Ovcar3 cell line) (A) hut not TAG-72-ve/low cancer target cells (MESOV cell line) (B). Target cells were allowed to adhere to xCELLigence Real-Time Cell Analysis (RTCA) plates overnight before addition of anti-TAG-72/CD3ϵ CRISPR FP T cells (green) at an effector to target ratio of 5:1. In parallel, NT control (purple), CD3ϵ RNP transfection alone (red) and lentivirally transduced anti-TAG-72 CAR-T cells (orange) were performed as controls. Cell impedance (represented as normalised cell index) was monitored over 20 h. Target cell proliferation under normal growth conditions (blue) was also monitored throughout. Biological duplicates were used where possible.

FIG. 11, Anti-CD19/CD3ϵ CRISPR FP T cells mediate potent cell killing of CD19+ target tumor cells (Ovcar3 cancer cell line engineered for stable expression of CD19). Anti-CD19/CD3ϵ CRISPR FP T cells were created via CRISPR KI of anti-CD19 scFv donor DNA [SEQ ID NO: 10] into CD3ϵ locus. Target cells were allowed to adhere to RTCA plates overnight before addition of anti-CD19/CD3ϵ CRISPR FP T cells (green) at an effector to target ratio of 5:1. In parallel, NT control (purple), CD3ϵ KO (red) and lentivirally transduced anti-CD19 CAR-T cells (orange) were performed as controls. Cell impedance (represented as normalised cell index) was monitored over 20 h. Target cell proliferation under normal growth conditions (blue) was also monitored throughout, Biological duplicates were used throughout, where CARs and FPs were generated using the T cells from two independent healthy donors. Intra-assay duplicates or triplicates were used.

FIG. 12, FACS analysis of surface expression of CD3 and TCRα/β in T cells 4 days after CD3ϵ and CD3γ RNP transfection, demonstrating the knock-out (KO) efficiency CD3ϵ and CD3γ RNPs. Panels A and F: non-transfected controls. Panels B and G: T cells treated with CD3δ gRN A-1 [SEQ ID NO: 3]. Panels C and H: T cells treated with CD3γ gRNA-1 [SEQ ID NO: 4]. Panels D and I: T cells treated with CD3γ gRNA-2 [SEQ ID NO: 5]. Panels E and J: T cells treated with CD3γ gRNA-3 [SEQ ID NO: 6]. Panels A to E: CD3γ staining. Panels F to J: TCRα/β staining. Transfection of CD3δCRISPR guide RNA [SEQ ID NO: 3] or CD 3γ CRISPR guide RNAs gRNA-1 [SEQ ID NO: 4], gRNA-2 [SEQ ID NO: 5] and gRNA-3 [SEQ ID NO: 6] formed Cas9 RNPs disrupts the CD3 and TCR receptor of human CD3+ T cells.

FIGS. 13. Creation of anti-TAG-72 CD3ϵ, CD3δ and CD3γ CRISPR FP T cells via transfection of CD3ϵ, CD3δ or CD3γ RNP with anti-TAG-72 scFv donor DNAs (with Flag epitope) [SEQ ID NO: 9, 11 or 12, respectively], with CRISPR knock-in of the anti-TAG-72 scFv at the N-terminal end of CD3ϵ, CD3d or CD3g molecules in human T cells, respectively. Cells were analysed for expression of Flag or TCRα/β by FACS 10 days after transfection. Panels A and F: Non-transfected T cells, Panels B and G: T cells transduced with anti-TAG-72 CAR lentiviral vectors, Panels C and H: T cells transfected with CD3ϵ RNP and 2 ug anti-TAG-72 scFv donor DNA. Panels D and I: T cells transfected with CD3δ RNP and 2 ug anti-TAG-72 scFv donor DNA. Panels E and J: T cells transfected with CD3γ RNP and 2 ug anti-TAG-72 scFv donor DNA). Panels A to E: Staining for Flag (surrogate for TAG-72 scFv). Panels F to J: Staining for TCRα/β and Flag (surrogate for TAG-72 scFv). Panel K: Summary of the anti-TAG-72 say surface expression level in primary human T cells from one donor, Robust Coefficient of Variation (R-CV) in the scFv+population (R-CV=100*1/2(intensity [at 84.13 percentile]−intensity [at 15.87 percentile])/median) was used to measure the dispersion of the scFv expression; Panel L: mean fluorescence intensity (MH) and R-CV of lentiviral anti-TAG-72 CAR+ T cells and anti-TAG-72/CD3ϵ CRISPR FP T cells from different donors (each dot represents an independent experiment).

FIGS. 14A and 14B. Anti-TAG-72 CD3δ and CD3γ CRISPR FP T cells mediate cell killing of TAG-72hi expressing target cells as potently as anti-TAG-72/CD3ϵ CRISPR FP T cells. CRISPR knock-in of TAG-72 scFv into either CD:3ϵ, CD3δ or CD3γ results in functional TFPs endowed with the ability to eliminate TAG-72hi expressing target cells (Ovcar3 ovarian cancer cell line) (A) but not TAG-72low expressing target cells (MESON cancer cell line) (B). Target cells were allowed to attach to RTCA plates for approximately 15 h before addition of anti-TAG-72 CD3ϵ (orange), CD3δ (black) or CD3γ (brown) CRISPR FP T cells. In parallel, NT control (purple), CD3ϵ KO (red) and lentiviral transduced anti-TAG-72 CAR-T cells (green) were performed as controls. Cell impedance (represented as normalised cell index) was monitored over 20 h. Target cell proliferation under normal growth conditions (blue) was also monitored throughout. Data represents the impact of CARs and TFPs generated using the T cells from a single healthy donor. Intra-assay triplicates were used, i.e. each sample was tested in triplicates to avoid well-to-well variation.

FIG. 15. Xenograft disease model—schematic procedure for the growth of human tumor in NOD SCID gamma (NSG) mice. NSG mice were subcutaneously administered 1×10e7 Ovcar3 tumor cells. At day 60, when the tumors grew to approximately 200 mm³, 2×5×10e6 TFP or CAR-T cells were adoptively transferred by intravenous injection. Tumor volume was measured twice a week until termination of the experiment.

FIG. 16. Tumor growth curve for mice treated with non-transfected T cells (NT), anti-TAG-72 CART cells (LV TAG-72), and anti-TAG-72/CD3ϵ CRISPR FP T cells (CRP CD3ϵ TAG-72). Data were presented as mean tumor volume±SEM at indicated time points (n≥4).

FIGS. 17A, 17B and 17C, Anti-CD19/TAG-72 dual targeting CRISPR FP T cells mediate potent and specific cell killing of tumor cells. CD3ϵ+δ or γ dual CRISPR FP T cells expressing both anti-CD19 and anti-TAG-72 scFv mediate cell killing of CD19hi target tumor cells (CD19 stable expressing Hela cancer cell line) (A) and TAG-72hi/CD19neg cells line Ovcar3 (A) but not CD19neg/TAG-72neg HeLa parental cell line (C). To test function, target cells were allowed to adhere to RTCA plates for 6 h before addition of anti-CD19/CD3ϵ CRISPR TFP T cells (green) or anti-CD19/CD3ϵ+anti-TAG-72/CD3δ dual CRISPR FP T cells (purple) or anti-CD19/CD3ϵ+anti-TAG-72/CD3γ dual CRISPR FP T cells (orange) at an effector to target ratio of 5:1. Co-culture of non-transfected T cells (NT) (red) at a comparable E:T ratio was performed in parallel. Cell impedance (represented as normalized cell index) was monitored over 20 h. Target cell proliferation under normal growth conditions (blue) was also monitored throughout. Intra-assay triplicates were used.

FIGS. 18A and 18B. Following continued antigen exposure, anti-CD19/CD3ϵ+anti-TAG-72/CD28 dual CRISPR FP T cells retain in vitro cytotoxicity and a heightened activation phenotype. A. Percent cytotoxicity (relative to target cells alone maintained under normal growth conditions) was determined following 20 h of co-culture, where target cells were maintained with anti-CD19/CD3ϵ CRISPR FP T cells (green) or with anti-CD19/CD3ϵ+anti-TAG-72/CD28 dual CRISPR FP T cells (purple) at an effector to target ratio of 1:1. In parallel, T cells (no CAR) (red) co-cultures were performed as controls. Data represent the impact of CRISPR FP T cells generated using the T cells from a single healthy donor in technical triplicate. B. T cell activation was determined by flow cytometry where the co-expression of CD25 and CD69 was assessed on T cells (no CAR) (Panel A), anti-CD19/CD3ϵ CRISPR FP T cells (Panel B) and anti-CD19/CD3ϵ+anti-TAG-72/CD28 dual (Panel C) CRISPR FP T cells following continued antigen exposure. Debris was excluded from analysis before selecting for single, viable cells. Data represents a single biological replicate. Values are presented as the frequency of viable cells.

FIG. 19. Generation of anti-TAG-72/2B4 CRISPR FP NK cells. Creation of anti-TAG-72/2B4 CRISPR FP NK cells via transfection of 2B4 RNP with anti-TAG-72 scFv donor DNAs (with Flag epitope), and knock-in of the anti-TAG-72 scFv at the N-terminal end of 2B4 molecules in NK-92 cells, a human NK cell line. Non-transfected (Panel A) and Transfected (Panel B) NK-92 cells were analyzed for surface expression of Flag or 2B4 by flow cytometry 3 days after transfection. The pre-sorted transfected NK-92 cells (Panel C) were sorted (Panel D) based on Flag expression to get the purified anti-TAG-72/2B4 CRISPR FP NK cells.

FIGS. 20A-20D, In vitro xCELLigence assays were performed to evaluate the killing potency of anti-TAG-72/2B4 CRISPR FP NK cells (green) on TAG-72hi Ovcar3 (A-B) and TAG-72low MESOV (C-D) cancer target cells at an E:T ratio of either 1:4 or 1:8. In parallel, non-transfected NK-92 (black) and Flag negative NK-92 fraction (orange) after sorting were performed as controls. Cell impedance (represented as normalized cell index) was monitored over 20 h. Target cell proliferation under normal growth conditions was also monitored throughout. Intra-assay triplicates were used.

FIG. 21. Generation of scFv and receptor CRISPR knock-in FP induced pluripotent stem cells (iPSCs) as a source of cells for adoptive cell therapy. Workflow of deriving scFv and receptor FP immune cells from iPSCs.

FIG. 22. PCR strategy for genotyping of the anti-TAG-72/CD3ϵ CRISPR FP iPSCs. Four sets of PCR primers flanking the left or right homological arms (HA-L, or HA-R) are chosen, which amplify wild-type or knock-in alleles (P1-F: CAATGTTCAAAATGGAGGCT, SEQ ID NO: 104; P1-R: GAACCGCTCGTIGTACTTG, SEQ ID NO: 105; P2-F: CAATGTTCAAAATGGAGGCT, SEQ ID NO: 106; P2-R: TACAAAGAATGATGGGGTGA, SEQ ID NO: 107; P3-F: CGGCGTGCCCGATAGATT, SEQ ID NO: 108; P3-R: ATTCTGGCTACGTCTCCT, SEQ ID NO: 109; P4-F: ATTTCTAGTTGGCGTTTGG, SEQ ID NO: 110; P4-R: ATTCTGGCTACGTCTCCT, SEQ ID NO: 111).

FIGS. 23A-23B. Morphology of non-transfected iPSC colonies (A) and anti-TAG-72/CD3ϵ CRISPR FP iPSC colonies at day 12 after CD3ϵ gRNA-1 RNP and anti-TAG-72 scFv donor DNA transfection (B).

FIG. 24. Genotyping PCR results of using flanking primer sets. Genomic DNA was extract from non-transfected iPSCs (NT) and anti-TAG-72/CD3ϵ CRISPR FP iPSCs for PCR amplification using the primers described previously. PCR products were visualized after 2% agrose gel electrophoresis with DNA Molecular-Weight (MW) markers.

### DETAILED DESCRIPTION

### Definitions

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout the specification and claims the terms ‘a’ and ‘an’ are to be taken to mean ‘at least one’ and are not to be taken as excluding “two or more” unless the context clearly dictates otherwise.

The term “cytotoxic cell” should be understood as a reference to any cell, in particular an immune cell, having receptor-based system with enhanced activity for a specific target, and able to kill a target cell. The term “immune cell” should be understood in its broadest sense and includes, without limitation, T cells, NKT cells, NK cells, B cells and phagocytes.

The term “cell receptor” is to be understood to include both type I and type II transmembrane proteins and also to include co-receptors such as DAP10, DAP12 and FcRγ. As described herein, a cell is modified in an endogenous gene encoding a cell receptor. However, the cell, before or after the modification, may not necessarily express the cell receptor. For example, in embodiments where a stem cell is modified in an endogenous gene encoding a cell receptor, the cell receptor is not expressed in the stem cell before or after the modification, but can be expressed in an immune cell differentiated from the stem cell. In embodiments where an immune cell is modified in an endogenous gene encoding a cell receptor, the immune cell may naturally express the cell receptor without a modification, and a modified immune cell may express a modified cell receptor.

The term “nucleic acid” molecule should be understood as a reference to both deoxyribonucleic acid and ribonucleic acid thereof. The subject nucleic acid molecule may be any suitable form of nucleic acid molecule including, for example, a genomic, cDNA or ribonucleic acid molecule. The nucleic acid molecule can be, without limiting, a natural nucleic acid, an artificial nucleic acid or chemically modified nucleic acid.

To this end, the term “expression” refers to the transcription of DNA or the translation of RNA resulting in the synthesis of a peptide, polypeptide or protein. A nucleic acid construct, for example, corresponds to the construct which one may seek to introduce or transfect into a cell.

The term “chimeric antigen receptor” (“CAR”) should be understood as a reference to engineered receptors which graft an antigen binding moiety onto an immune effector cell. Typically, these receptors are used to graft the specificity of a monoclonal antibody onto a T cell, with transfection of their coding sequence facilitated by retroviral vectors. One common form of these molecules are fusions of single-chain variable fragments (scFv) derived from monoclonal antibodies, fused to a CD3-zeta chain transmembrane and endodomain. Such molecules result in the transmission of a CD3-zeta chain signal in response to recognition by the scFv of its target. When T cells express this chimeric molecule, they recognize and kill target cells that express the antigen to which the scFv is directed.

The term “antigen recognition moiety” should be understood as a reference to a domain, e.g., an extracellular portion of a receptor, which recognises and binds to a target antigen of interest, that is, a target specific binding element. The antigen recognition moiety is usually, but not limited to, an scFv.

The term “linker” refers to any oligo- or polypeptide that functions to link two polypeptide sequences, e.g., to link the extracellular domain of a cell surface receptor to an antigen recognition moiety for a targeted antigen.

The terms “target antigen” should be understood as a reference to any proteinaceous or non-proteinaceous molecule expressed by a cell which may be targeted by the receptor-expressing immune cells such as T cells, NKT cells or NK cells of the present invention. It would be appreciated that these are molecules which may be “self” molecules in that they are normally expressed in the body of a patient (such as would be expected on some tumor cells or an autoreactive cells) or they may be non-self molecules such as would be expected where a cell is infected with a microorganism (e.g., viral proteins). It should also be understood that the subject antigen is not limited to antigens (whether self or not) which are naturally able to elicit a T or B cell immune response. Rather, in the context of the present invention, reference to “antigen”, “antigenic determinant” or “target antigen” is a reference to any proteinaceous or non-proteinaceous molecule to be targeted. As detailed hereinbefore, the target molecule may be one to which the immune system is naturally tolerant, such as a tumor antigen or auto-reactive immune cell antigen. However, it may be desirable (even in light of potential collateral damage) to nevertheless target this antigen, for example to minimize the potentially even more severe side effects which might be observed with a highly non-specific and systemic treatment, such as chemotherapy or immunosuppression, or to reduce the duration of treatment via a highly targeted treatment and/or to maximise the prospect of killing all unwanted cells. Preferably, said molecule is expressed on the cell surface.

The terms “neoplastic, condition” should be understood as a reference to a condition characterised by the presence or development of encapsulated or unencapsulated growths or aggregates of neoplastic cells. Reference to a “neoplastic cell” should be understood as a reference to a cell exhibiting abnormal growth. The neoplastic cells comprising the neoplasm may be any cell type, derived from any tissue, such as an epithelial or non-epithelial cell. The term “neoplasm” should be understood as a reference to a lesion, tumor or other encapsulated or unencapsulated mass or other form of growth or cellular aggregate which comprises neoplastic cells. In the context of the present invention, neoplasm or neoplastic condition should be understood to include reference to all types of cancerous growths or oncogenic processes, metastatic tissues or malignantly transformed cells, tissues or organs irrespective of histopathologic type or state of invasiveness.

The term “growth” should be understood in its broadest sense and includes reference to enlargement of neoplastic cell size as well as proliferation. The phrase “abnormal growth” in this context is intended as a reference to cell growth which, relative to normal cell growth, exhibits one or more of an increase in individual cell size and nuclear/cytoplasmic ratio; an increase in the rate of cell division; an increase in the number of cell divisions a decrease in the length of the cell division cycle; an increase in the frequency of periods of cell division or uncontrolled proliferation; and evasion of apoptosis. Without limiting the present invention in any way, the common medical meaning of the term “neoplasia” refers to “new cell growth” that results as a loss of responsiveness to normal growth controls, e.g., to neoplastic cell growth. Neoplasias include “tumors” which may be benign, pre-malignant or malignant.

The term “carcinoma” is recognised by those skilled in the art and refers to malignancies of epithelial or endocrine tissues including respiratory system carcinomas, gastrointestinal system carcinomas, genitourinary system carcinomas, testicular carcinomas, breast carcinomas, prostate carcinomas, endocrine system carcinomas and melanomas. The term also includes carcinosarcomas, e.g. which include malignant tumors composed of carcinomatous and sarcomatous tissues. An “adenocarcinoma” refers to a carcinoma derived from glandular tissue or in which the tumor cells form recognisable glandular structures. Reference to the terms “malignant neoplasm” and “cancer” and “carcinoma” herein should be understood as interchangeable.

The term “treatment” does not necessarily imply that a mammal is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity of the onset of a particular condition. “Treatment” may also reduce the severity of an existing condition.

Unless otherwise defined, all technical and scientific terms used herein have the same meaning as commonly understood by one of ordinary skill in the art to which this invention belongs.

This disclosure provides modified immune cells in which cell surface receptors have been modified to recognize one or more target antigens, in particular tumor-associated antigens. This disclosure also provides a method of making modified immune cells (e.g., cytotoxic cells) targeted against one or more target antigens, in particular tumor-associated antigens, by editing the cell receptors, in particular cell surface receptors expressed by immune cells such as T cells or NK cells, while retaining the natural receptor expression, assembly and signalling mechanisms. The present method is simple and allows for efficient generation of a homogenous population of modified immune cells expressing modified cell surface receptors targeted against one or more target antigens. Further, this disclosure provides stem cells modified in one or more endogenous genes encoding cell surface receptors and capable of differentiating into immune cells expressing modified cell surface receptors that recognize one or more target antigens, as well as methods of making such modified stem cells and methods of making immune cells from such modified stem cells.

### Immune Cells

### T Cells

Reference to a “T cell” should be understood as a reference to any cell comprising a T cell receptor. In this regard, the T cell receptor may comprise any one or more of the α, β, γ or δ chains. As would be understood by the person of skill in the art, NKT cells also express a T cell receptor and therefore target antigen specific NKT cells can also be generated according to the present invention. The present invention is not intended to be limited to any particular sub-class of T cell, although in one embodiment the subject T cell expresses an α/β TCR dimer. In some embodiments, said T cell is a CD4+ helper T cell, a CD8+killer T cell, or a NKT cell. Without limiting the present invention to any one theory or mode of action, CD8+ T cells are also known as cytotoxic cells. As a major part of the adaptive immune system, CD8+ T cells scan the intracellular environment in order to target and destroy, primarily, infected cells. Small peptide fragments, derived from intracellular content, are processed and transported to the cell surface where they are presented in the context of MHC class I molecules. However, beyond just responding to viral infections, CD8+ T cells also provide an additional level of immune surveillance by monitoring for and removing damaged or abnormal cells, including cancers. CD8+ T cell recognition of an MHC I presented peptide usually leads to either the release of cytotoxic granules or lymphokines or the activation of apoptotic pathways via the FAS/FASL interaction to destroy the subject cell. CD4+ T cell, on the other hand, generally recognise peptide presented by antigen presenting cells in the context of MHC class II, leading to the release of cytokines designed to regulate the B cell and/or CD8 + T cell immune responses.

### Natural Killer T Cells

Natural killer cells T (also called NKT or T/NK cells) are a specialised population of T cells that express a semi-invariant T cell receptor (TCR αβ) and surface antigens typically associated with natural killer cells. The TCR on NKT cells is unique in that it commonly recognizes glycolipid antigens presented by the MHC I-like molecule CD1d. Most NKT cells express an invariant TCR alpha chain and one of a small number of TCR beta chains. The TCRs present on type I NKT cells commonly recognise the antigen alpha-galactosylceramide (alpha-GalCer). Within this group, distinguishable subpopulations have been identified, including CD4⁺CD8⁻ cells, CD4⁻CD8⁺ cells and CD4⁻CD8⁻ cells. Type II NKT cells (or noninvariant NKT cells) express a wider range of TCR α chains and do not recognise the alpha-GalCer antigen. NKT cells produce cytokines with multiple, often opposing, effects, for example either promoting inflammation or inducing immune suppression including tolerance. As a result, they can contribute to antibacterial and antiviral immune responses, promote tumor-related immunosurveillance, and inhibit or promote the development of autoimmune diseases. Like natural killer cells, NKT cells can also induce perforin-, Fas-, and TNF-related cytotoxicity. Accordingly, reference to the genetically modified T cells of the present invention should be understood to include reference to NKT cells.

### NK Cell

Natural killer (NK) cells are a type of cytotoxic lymphocyte that form part of the innate immune system. NK cells provide rapid responses to virus-infected cells, acting at around 3 days after infection, and also respond to tumor formation. Typically, immune cells such as T cells detect major histocompatibility complex (MHC) presented on infected or transformed cell surfaces, triggering cytokine release and resulting in lysis or apoptosis of the target cell. NK cells, however, have the ability to recognize stressed cells in the absence of antibodies or MHC, allowing for a much faster immune reaction. This role is especially important because harmful cells that are missing MHC 1 markers cannot be detected and destroyed by other immune cells, such as T cells. In contrast to NKT cells, NK cells do not express TCR or CD3 but they usually express the surface markers CD16 (FcγRIII) and CD56.

In some embodiments, the immune cell is a T cell, an NKT cell or an NK cell.

In other embodiments, the immune cell is a B cell or a macrophage.

### Stem Cells

Stem cells can serve as an alternative cell source for the modification described herein, and can be stored in cell banks and provide the starting point for allogeneic “off-the-shelf” cell products.

The term “stem cell” should be understood as a reference to any cell which exhibits the potentiality to develop in the direction of multiple (i.e., two or more) lineages of cells, and includes, for example, embryonic stem cells, adult stem cells, umbilical cord stem cells, haemopoietic stem cells (HSCs), totipotent cells, progenitor cells, precursor cells, pluripotent cells, multipotent cells, or de-differentiated somatic cells (such as an induced pluripotent stem cells, or “iPSCs”). By “pluripotent” is meant that the subject stem cell can differentiate to form, inter alia, cells of any one of the three germ layers, these being the ectoderm, endoderm and mesoderm.

In some embodiments, the subject stem cell is an induced pluripotent stem cell (iPSC).

In some embodiments, stem cells are genetically modified via the present method to generate modified stem cells containing a modified cell receptor gene. In some embodiment, modified stem cells are cultured to differentiate into immune cells which express a modified cell receptor comprising the antigen recognition moiety in the extracellular domain of the cell receptor.

Methods for differentiating iPSCs into immune cells, in particular into T-cells or NK cells, are known in the art (Themeli et al., Nat Biotechnol, 2013. 31(10): p. 928-33; Maeda et al., Cancer Res, 2016. 76(23): p. 6839-6850; Li et al., Cell Stem Cell, 2018. 23(2): p. 181-192 e185).

### Cell Receptors

Immune cells and, in particular, T cells and NK cells, express a wide range of cell surface receptors that are associated with cellular activation and function, such as cytotoxic activity. Examples of such receptors expressed on T cells include, but are not limited to, CD2, CD3, CD4, CD8, TCR, CD28, IL-2Rα, IL-15Rα, IL-21R and 2B4, CD69, CD27, DNAM1, IL-12R, IL-18R . Examples of such NK cell receptors include, but are not limited to NKG2D, CD94, CD16, NKp46, NKp30, NKp44, 2B4, IL-2Rα, IL-15Rα, IL-21R, CD69, CD27, DNAM1, DAP10, DAP12, FcRγ, 1L-12R, and IL-18R.

### T Cell Receptor (TCR)

Reference to a “T cell receptor” (TCR) should be understood as a reference to a heterodimer found on the surface of T cells or NKT cells which recognise peptides presented by MHC. Specifically, CD8+ T cells recognise peptide presented in the context of MHC class I while CD4+ T cells recognise peptide presented in the context of MHC class II.

The TCR is composed of two subunits, both members of the immunoglobulin superfamily. In most (˜95%) of T cells, the subunits are the α and β subunits; in the minority they are the γ and δ subunits. The two subunits form a disulphide-linked heterodimer and, together, recognize “foreign” peptide antigen presented by class I HLA molecules.

The γ, δ, α and β chains are composed of two domains in the extracellular segment (or two subdomains in the extracellular domain): a variable (V) region and a constant (C) region, which both form part of the immunoglobulin superfamily and which fold to form antiparallel β-sheets. The constant region is proximal to the cell membrane, followed by a transmembrane region and a short cytoplasmic tail, while the variable region binds to the peptide/MHC complex.

The variable domains of both the TCR α-chain and β-chain each express three hypervariable or complementarity determining regions (CDRs), whereas the variable region of the β-chain has an additional area of hypervariability (HV4) that does not normally contact antigen and, therefore, is not considered a CDR. The processes for the generation of TCR diversity are based mainly on genetic recombination of the DNA encoded segments in precursor T cells—either somatic V(D)J recombination using RAG1 and RAG2 recombinases or gene conversion using cytidine deaminases. Each recombined TCR possesses unique antigen specificity, determined by the structure of the antigen-binding site formed by the α and β chains, in the case of αβ T cells, or γ and δ chains in the case of γδ T cells. The TCR α chain is generated by VJ recombination, whereas the β chain is generated by VDJ recombination. Likewise, generation of the TCR γ chain involves VJ recombination, whereas generation of the TCR δ chain occurs by VDJ recombination. The intersection of these specific regions (V and J for the α or γ chain; V, D, and J for the β and δ chain) corresponds to the CDR3 region that is important for peptide/MHC recognition. It is the unique combination of the segments at this region, along with palindromic and random nucleotide additions, which account for the even greater diversity of T cell receptor specificity for processed antigenic peptides.

The αβ and γδ structures form the core “recognition” structure the TCR. However, TCR signalling requires a complex of TCR αβ or TCR γδ together with the CD3 co-receptor. CD3 is a complex of four separate, but related molecules: one CD3γ chain, one CD3δ and two CD3ϵ chains, All the aforementioned TCR complex components are surface-exposed, membrane-bound molecules. The final component of the TCR complex is an internal (but membrane-bound) signalling molecule—the ζ chain. It is responsible for activation of the T cell upon engagement of the TCR.

Accordingly, reference to a cytotoxic cell targeted against a target antigen should be understood as a cell for which a receptor gene has undergone modification to encode a modified receptor that exhibits specificity for a target antigen

### NK Cell Receptor

The receptors expressed by NK cells can vary from cell to cell, depending on a number of factors including the stage of cell maturation. NK cells do not express TCR or CD3 but they usually express the surface markers CD16 (FcγRIII) and CD56. They can also express cell receptors such as NKG2D, CD94, NKp46, NKp30, NKp44 and 2B4.

### Target Antigen

It would be understood by the skilled person that in the context of TCR binding, the target antigen will take the form of a peptide derived from an antigen, which peptide is expressed in the context of either MHC I or MHC II. It should be understood that the target antigen may be any molecule expressed by the cell to be targeted. That is, the molecule which is targeted may be exclusively expressed by the target cell or it may also be expressed by non-target cells too. Preferably, the target antigen is a non-self target antigen or a target antigen which is otherwise expressed exclusively, or at a significantly higher level than by normal cells, by the cells to be targeted. However, depending on the disease condition to be treated, it may not always be possible to identify and target a non-self target antigen.

As would be appreciated by the skilled person, the identification of antigens which are exclusive to tumors is a significant area of research, but in respect of which there has been limited progress. Since tumor cells are usually self cells, (as opposed to, for example, tumors arising from transplant tissues), it is the case that the antigens which they express are not only self antigens, but are likely to also be expressed by at least some of the non-neoplastic cells of the tissue from which the tumor is derived. This is clearly a less than ideal situation due to the side-effects (in terms of destruction of non-neoplastic tissue) which can arise when an anti-neoplastic treatment regime is targeted to such an antigen, but is unavoidable. Nevertheless, some progress has been made in terms of identifying target tumor antigens which, even if not expressed exclusively by tumor cells, are expressed at lower levels or otherwise less frequently on non-neoplastic cells.

Non-limiting examples of antigens include the following: Differentiation antigens such as MART-1/MelanA (MART-I), gp100 (Pmel 17), tyrosinase, TRP-1, TRP-2 and tumor-specific multilineage antigens such as, MAGE-1, MAGE-3, BALE, GAGE-1, GAGE-2, p15; overexpressed glycoproteins such as MUC1 and MUC16; overexpressed embryonic antigens such as CEA; overexpressed oncogenes and mutated tumor-suppressor genes such as p53, Ras, HER-2/neu; unique tumor antigens resulting from chromosomal translocations, such as BCR:-ABL, E2A-PRL, H4-RET, IGH-IGK, MYL-RAR; viral antigens, such as the Epstein Barr virus antigens EBVA and the human papillomavirus (HPV) antigens E6 and E7. Other tumor associated antigens include: folate receptor alpha (FRa), EGFR, CD47, TSP-180, MAGE-4, MAGE-5, MAGE-6, RAGE, NY-ESO, pl 85erbB2, pl80erbB-3, cMet, nm-23H1, PSA, CA 19-9, CAM 17.1, NuMa, K-ras, beta-catenin, CDK4, Mum-1, p 15, p 16, 43-9F, 5T4, 791Tgp72, alpha-fetoprotein, beta-HCG, BCA225, BTAA, CA 125, CA 15-3\CA 27. 29\BCAA, CA 195, CA 242, CA-50, CAM43, CD68\P1, CO-029, FGF-5, G250, Ga733\EpCAM, HTgp-175, M344, MA-50, MG7-Ag, NB/70K, NY-CO-1, RCAS 1, SDCCAG16, TA-90\Mac-2 binding protein\cyclophilin C-associated protein, TAAL6, TAG-72, TLP, TPS, PSMA, mesothelin and BCMA.

In some embodiments, the target antigen is a tumor-associated antigen, in particular a protein, glycoprotein or a non-protein tumor-associated antigen.

In some embodiments, the target antigen is a tumor-associated antigen, in particular the tumor-associated antigen is TAG-72.

In other embodiments, the target antigen is a surface protein that can be used for tumor-targeting, in particular the target antigen is CD19 or CD20.

In other embodiments the target antigen is a viral protein, in particular a viral protein that is expressed on the surface of virally-infected cells. Examples include the envelope (env) proteins of a range of enveloped virus; the spike protein of coronaviruses; the hemagglutin (HA) and neuraminidase proteins of influenza virus, the membrane fusion proteins of viruses including HIV, ebola, RSV, HCV, lasso, coronaviruses and others. In some embodiments the target antigen is a cell surface receptor or co-receptor that serves as the site for viral binding to a cell. Examples include CD4 and CCR5 in the case of HIV and ACE2 receptor in the case of SARS-CoV-2.

### Antigen Recognition Moiety

In some embodiments, the antigen recognition moiety for the targeted antigen comprises an antibody fragment, including but not limited to scFv, Fv, Fab, single domain antibody (SdAb), homodimeric heavy-chain antibody (HCAb), diabody, single variable domain, nanobody, VhH domain and V-NAR domain.

In some embodiments, the antigen recognition moiety is comprised of a scFv directed to a target antigen described herein. In some embodiments the antigen recognition moiety is comprised of an scFv directed to CD19 or TAG-72.

Antigen recognition moiety used herein is included as part of a modified cell surface receptor, for example, operably linked to or inserted within the extracellular domain of an unmodified (native) cell surface receptor, in some embodiments, an antigen recognition moiety is operably inserted within the extracellular domain of a cell surface receptor at a position close to the free end of the extracellular domain, for example, within 5, 4, 3, 2 or 1 amino acid(s) from the free end amino acid residue of the extracellular domain of the cell surface receptor in such embodiments, the extracellular domain of the native cell receptor remains substantially intact (except for the insertion of the antigen recognition moiety close to the free end of the extracellular domain), and the transmembrane and intracellular domains are kept intact. In some embodiments, an antigen recognition moiety is operably linked to the free end amino acid residue of the extracellular domain of a cell surface receptor—in such embodiments, except for the addition of the antigen recognition moiety to the free end of the extracellular domain, the extracellular, transmembrane and intracellular domains of the native cell receptor are kept unchanged. In some embodiments the free end of the extracellular domain is the extracellular free end of a transmembrane protein, and in some embodiments the extracellular free end is at the N-terminus or the C-terminus of the transmembrane protein.

In some embodiments, the linkage between an antigen recognition moiety and an amino acid of the extracellular domain of a cell receptor is made through a linker, i.e., a short oligo- or polypeptide linker that forms the linkage between the antigen recognition moiety and an extracellular domain amino acid of a cell receptor. A linker can be as short as a few amino acids in length (e.g., 3, 4, 5, 6 or 7 amino acids), or longer (e.g., 10, 15, 20, 25 or 30 amino acids). Typically, the linker consists of amino acids of a small size, e.g., Gly (G), Ala (A), or Ser (S). In one embodiment the linker is a (G4S)3 linker.

### Modified Immune Cells

Disclosed herein is a modified immune cell that recognizes a target antigen. The genome of the modified immune cell comprises a nucleic acid sequence encoding an antigen recognition moiety for the target antigen, inserted in an endogenous cell receptor gene to form a modified cell receptor gene. The insertion is at a specific site in the coding region of the endogenous cell receptor gene such that the endogenous (i.e., native) cell receptor is modified to include the antigen recognition moiety in the extracellular domain, and expression of the modified cell receptor gene is under control of the endogenous cis-regulatory elements (including, e.g., the promoter, any enhancer, the 3′ untranslated region, and the polyyadenylation signal) at the endogenous cell receptor gene locus.

Also disclosed herein is a modified immune cell which comprises two or more modified cell receptor genes and recognizes two or more different target antigens.

Immune cells referenced herein include, for example, T cells, NKT cells, NK cells, B cells and macrophages.

In one embodiment, a modified immune cell is a modified T cell and the cell receptor that has been modified is a cell receptor naturally expressed by the T cell. Examples of cell receptors naturally expressed by a T cell are CD2, CD3 (any one of CD3γ, CD3δ and CD3ϵ), CD4, CD8, TCR, and CD28. In some embodiments, a modified immune cell is a T cell which comprises two or more modified cell receptor genes and recognizes two or more different target antigens, for examples, two or more target antigens with at least one target antigen being a tumor antigen. In some embodiments, two different CD3 subunit genes (i.e., two selected from a CD3γ gene, a CD3δgene, or a CD3ϵ gene) have been modified and the resulting modified T cell expresses two modified CD3 chains that recognize two different target antigens. In some embodiments, a CD3 subunit gene and a CD28 gene have been modified and the resulting modified T cell expresses a modified CD3 receptor that recognizes a target antigen and a modified CD28 receptor that recognizes another target antigen.

In another embodiment, a modified immune cell is a modified NK cell and the cell receptor that has been modified is a cell receptor naturally expressed by the NK cell. Examples of cell receptors naturally expressed by a NK cell include NKG2D, CD94, NKp46, NKp30, NKp44, and 2B4,

In still another embodiment, a modified immune cell is a modified B cell and the cell receptor that has been modified is a cell receptor naturally expressed by the B cell.

Further disclosed herein is a cell population that comprises modified immune cells disclosed herein. In some embodiments, the modified immune cells constitute at least 5%, 10%, 15%, 20%, 25%, 30%, 35%, 40%, or more of the cells in the cell population.

### Nucleic Acid Molecules

Disclosed herein is a nucleic acid construct, also referred to as a targeting nucleic acid construct. The construct comprises a nucleic acid sequence encoding an antigen recognition moiety that recognizes a target antigen, flanked by a 5′ homology arm and a 3′ homology arm. The 5′ and 3′ homology arms are homologous to the nucleotide sequences upstream and downstream of a specific site in the coding region of an endogenous cell receptor gene in an immune cell, and mediate insertion of the nucleic acid sequence into the site to form a modified cell receptor gene. As a result of the insertion at the specific site, the endogenous cell receptor is modified to include the antigen recognition moiety in the extracellular domain, and expression of the modified cell receptor gene is under control of the endogenous cis-regulatory elements at the endogenous cell receptor gene locus.

In some embodiments, the nucleic acid sequence encoding an antigen recognition moiety is not more than 2.5 kb, 2.0 kb, 1.9 kb, 1.8 kb, 1.7 kb, 1.6 kb, 1.5 kb, 1.4 kb, 1.3 kb, 1.2 kb, 1.1 kb or 1.0 kb.

The 5′ and 3′ homologous arms are of a length sufficient to mediate homologous recombination and integration of the nucleic acid sequence into a specific site. In some embodiments, the 5′ and 3′ homologous arms are each at least about 40 bp, 50 bp, 60 bp, 70 bp, 80 bp, 90 bp, 100 bp, 200 bp, 300 bp, 400 bp, 500 bp or 600 bp in length. In some embodiments, the 5′ and 3′ homologous arms are not more than 2 kb, 1.9 kb, 1.8 kb, 1.7 kb, 1.6 kb, 1.5 kb, 1.4 kb, 1.3 kb, 1.2 kb, 1.1 kb, 1 kb, 900 bp, 800 bp, 700 bp, 600 by each. In some embodiments, the 5′ and 3′ homologous arms are each about 200-600 bp in length, e.g., about 300 bp in length.

The nucleic acid sequence encoding an antigen recognition moiety can be derived from any human or non-human source. Non-human sources contemplated by the present invention include primates, livestock animals (e.g., sheep, pigs, cows, goats, horses, donkeys), laboratory test animal (e.g., mice, hamsters, rabbits, rats, guinea pigs), domestic companion animal (e.g., dogs, cats), birds (e.g., chicken, geese, ducks and other poultry birds, game birds, emus, ostriches) captive wild or tamed animals (e.g., oxen, kangaroos, dingoes), reptiles, fish, insects, prokaryotic organisms or synthetic nucleic acids.

It should be understood that the targeting nucleic acid constructs disclosed herein may comprise nucleic acid material from more than one source. For example, whereas the construct may originate from a particular microorganism, in modifying that construct to introduce the features defined herein, nucleic acid material from other microorganism sources may be introduced. These sources may include, for example, bacterial DNA (e.g., IRES DNA), mammalian DNA (e.g., the DNA encoding an scFv) or synthetic DNA (e.g., to introduce specific restriction endonuclease sites).

In some embodiments, the nucleic acid construct disclosed herein does not include a viral nucleotide sequence.

The nucleic acid construct can be double-stranded (ds) DNA, including linearized and cyclized dsDNA, or single-stranded DNA.

In one embodiment, the nucleic acid construct is purified linearized double-stranded DNA derived from high-fidelity PCR or linearized single-stranded DNA derived from PCR and enzyme reaction.

### Gene Delivery Method

CD8+ T cells (also known as cytotoxic killer T cells) are very effective in killing cells that they recognize through the TCR. Target cells for killer T cells include tumor cells and viral-infected cells. A number of investigators have explored (and still are exploring) the development of therapies based on the CA: Viva expansion of either donor or patient-derived killer T cells (Sadelain et al., Nature (2017) 545(7655): p. 423-431). While it is possible to produce large numbers of such cells, a significant limitation of this approach is the availability of T cells with the desired TCR specificity. For some common viral diseases (e.g. EBV) this is not a problem. However, tumor cells are largely “invisible” to the immune system and so it is much more difficult to obtain tumor-specific killer T cells. In addition, because the TCR only recognizes peptide antigens (in the context of class I HLA molecules), naturally-occurring killer T cells are incapable of recognizing non-peptide tumor antigens.

To try and address these limitations, a large body of work has been undertaken in the development of artificial or modified TCRs. One form of artificial TCR is the chimeric antigen receptor (CAR), which fundamentally consists of an antibody (scFv) recognition sequence attached to one or more T cell signalling sequences. Expression of a CAR by a T cell provides the T cell with the specificity directed by the scFv. The design, construction and use of CAR-T cells as cancer therapy is well known in the art (Kershaw et al., Nat Rev Cancer (2013) 13(8): p. 525-41; Sadelain et al., Nature (2017) 545(7655): p. 423-431). Despite their success in selected (and relatively rare) blood-based tumors, CAR-T cells have not yet been shown to be effective in solid tumors. One possible reason is the non-natural CAR signalling mechanism, which bypasses the evolution-selected, natural TCR mechanism.

An artificial TCR that more closely matches the natural TCR is the TCR fusion protein (TFP) approach of Baeuerle et al. (PCT/US2016/033146 by TCR2 Therapeutics, published as WO2016/187349). This involves fusing a scFv antigen recognition sequence to the N-terminal region of one of the molecules of the TCR complex. In doing so, the specificity of the complex is changed to the specificity of the scFv. The signalling mechanism remains unchanged. The manner in which the TFP cells are generated, however, remains very similar to generation of CAR-T cells. Basically, this involves creation of a transgene construct for the fusion protein; packaging of the construct into a lentivirus vector; transduction of the vector into donor T cells; selection and expansion of transduced cells that have incorporated the fusion protein construct, This method is complex and expensive, and results in a non-homogeneous product (due to the random insertion of lentiviral-delivered transgenes).

A method is provided herein for generating a modified immune cell that recognizes a target antigen by generating a modified cell receptor gene. The method comprises inserting a nucleic acid sequence encoding an antigen recognition moiety for the target antigen into an endogenous cell receptor gene in an immune cell to form a modified cell receptor gene. The insertion is at a specific site in the coding region of the endogenous cell receptor gene such that the endogenous cell receptor is modified to include the antigen recognition moiety in the extracellular domain, and wherein expression of the modified cell receptor gene is under control of the endogenous cis-regulatory elements at the endogenous cell receptor gene locus.

A method is also provided herein generating a cell population comprising modified immune cells that recognize a target antigen. The method comprises introducing to a population of immune cells, a nucleic acid sequence encoding an antigen recognition moiety for the target antigen, for insertion into an endogenous cell receptor gene in the immune cells to form a modified cell receptor gene, and obtaining a cell population, wherein at least a portion of the cells in the cell population are modified immune cells that comprise the modified cell receptor gene. The insertion is at a specific site in the coding region of the endogenous cell receptor gene such that the endogenous cell receptor is modified to include the antigen recognition moiety in the extracellular domain, and expression of the modified cell receptor aerie is under control of the endogenous cis-regulatory elements at the endogenous cell receptor gene locus.

In some embodiments of the present methods, the insertion is an in-frame insertion within the extracellular domain-coding sequence in an endogenous cell receptor gene, and is within 5 codons from the codon encoding the free end amino acid of the extracellular domain of the endogenous cell receptor. In some embodiments, the insertion is an in-frame insertion immediately following or followed by the codon encoding the free end amino acid of the extracellular domain of the endogenous cell receptor.

The present methods allow for generation of a cell population comprising modified immune cells with improved homogeneity. In some embodiments, the modified immune cells constitute at least 5%, 10%, 15%, 20%, 25%, 30%, 35%, 40%, or more of the cells in the cell population.

In some embodiments, the modified immune cells made by the present methods are cytotoxic cells targeted against at least one target antigen, particularly T cells or NK cells.

In some embodiments, the cell population made by the present method is a cell population of modified T cells with improved homogeneity.

In some embodiments, the modified T cells comprise a modified CD3 subunit gene (e.g., one of a CD3γ gene, a CD3δ gene and a CD3ϵ gene). In some embodiments, the modified T cells each comprise a modified CD3ϵ gene, wherein the modified CD3ϵ receptor includes an antigen recognition moiety for TAG-72 at the free end of the extracellular domain of the CD3ϵ receptor. In some embodiments, the modified T cells comprise two or more modified cell receptor genes and recognize two or more different target antigens (for example, at least one of the two target antigens being a tumor antigen). In some embodiments, at least two different CD3 subunit genes (i.e., two or more selected from a CD3γ gene, a CD3δgene or a CD3ϵgene) have been modified and the resulting modified T cells expresses at least two modified CD3 chains that recognize at least two different target antigens. In some embodiments, one or more CD3 subunit genes and a non-CD3 receptor gene (e.g., CD28 gene) have been modified and the resulting modified T cells express one or more modified CD3 receptors and a modified non-CD3 receptor that together recognize at least two target antigens.

### Non-Viral Methods

Methods of introducing a nucleic acid into a cell include physical, chemical, or biological means.

Physical methods for introducing a polynucleotide into a host cell include calcium phosphate precipitation, lipofection, particle bombardment, microinjection, electroporation, and the like.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle is a liposome an artificial membrane vesicle).

In one embodiment, a nucleic acid sequence is introduced into a host cell is by electroporation.

In some embodiments, a nucleic acid sequence is introduced into a host cell via a non-viral system; i.e., the delivery of the nucleic acid sequence into the host cell does not involve the use of a virus.

A number of non-viral based systems have been developed for gene transfer into mammalian cells. They include, hut are not limited to, CRISPR (i.e. CRISPR/Cpf1, CRISPR/Cas9, etc.), TALEN, transposon-based, ZEN, meganuclease, or Mega-TAL, among others.

In one embodiment, a modified cell receptor gene is generated by inserting a nucleic acid sequence at a specific site in in the coding region for the endogenous cell receptor gene, wherein the insertion is directed by CRISPR/Cas9 system.

### CRISPR/Cas9 System

The CRISPR (Clustered Regularly Interspaced Short Palindromic Repeats)/Cas9 system is an important component of the bacterial immune system that allows bacteria to recognize and destroy phages. In genome engineering applications, Cas9 endonuclease is targeted by guide RNA (tRNA) sequence homology to a given locus, where it induces a double stranded break (DSB) (Cong et al., Science (2013) 339(6121): p. 819-23). The resulting DSB is then repaired by one of two general repair pathways: the Non-Homologous End Joining (NHEJ) pathway and the Homology Directed Repair (HDR) pathway. The NHEJ repair pathway is the most active repair mechanism, capable of rapidly repairing DSBs, but frequently results in small nucleotide insertions or deletions (indels) at the DSB site, resulting in about a two-thirds chance of causing a frameshift mutation to knock-out a functional gene. The HDR pathway is less efficient but with high-fidelity. It uses longer homologous DNA arms to repair DNA lesions. It allows insertion of large gene inserts through introducing ssDNA or dsDNA donor DNA. consisting of two homological arms and a gene expression cassette, into cells along with RNPs. However, the HDR efficiency is generally low (Addgene, CRISPR 101: A Desktop Resource (2nd Edition), 2017.

Examples of guide RNA sequences designed for editing various cell receptors are provided in the following table.

TABLE 1

| | Gene | | |
|---|---|---|---|
| Receptor | Official | Genomic | |
| Name | Symbol | RefSeqGeue | Guide RNA Sequences Designed |
| CD3epsilon | CD3E | NG_007383.1 | GTTGGCGTTTGGGGGCAAGA (SEQ ID NO: 1) |
| | | | ATTTTCTAGTTGGCGTTTGG (SEQ ID NO: 2) |
| CD3delta | CD3D | NG_009891.1 | CCTCTATAGGTATCTTGAAG (SEQ ID NO: 3) |
| | | | TCCTCTATAGGTATCTTGAA (SEQ ID NO: 26) |
| | | | TTCCTCTATAGGTATCTTGA (SEQ ID NO: 27) |
| CD3gama | CD3G | NG_007566.1 | TCTCCTACCTTTGATTGACT (SEQ ID NO: 4) |
| | | | ACTTTGGCCCAGTCAATCAA (SEQ ID NO: 5) |
| | | | TGGCCCAGTCAATCAAAGGT (SEQ ID NO: 6) |
| CD3zeta | CD247 | NG_007384.1 | AGCTTTATCTCTTGGCACAG (SEQ ID NO: 28) |
| | | | GGCACAGAGGCACAGAGCTT (SEQ ID NO: 29) |
| TCRalpha | TRAC | NG_001332.3 | CTCTCAGCTGGTACACGGCA (SEQ ID NO: 30) |
| | | | AGAGTCTCTCAGCTGGTACA (SEQ ID NO: 31) |
| | | | GCTGGTACACGGCAGGGTCA (SEQ ID NO: 32) |
| TCRbeta | TRBC1 | NG_001333.2 | AGGTCGCTGTGTTTGAGCCA (SEQ ID NO: 33) |
| | TRBC2 | | AGGTCGCTGTGTTTGAGCCA (SEQ ID NO: 34) |
| TCRgama | TRGC1 | NG_029035.1 | CATCAAGTTGTTTATCTATG (SEQ ID NO: 35) |
| | TRGC2 | NG_001336.2 | GCATCAAGTTGTTTATCTAT (SEQ ID NO: 36) |
| | | | TGCATCAAGTTGTTTATCTA (SEQ ID NO: 37) |
| TCRdelta | TRDC | NG_001332.3 | AATAAGTTGATTATATTTGC (SEQ ID NO: 38) |
| | | | AAAACGGTUGGTTTGGTATG (SEQ ID NO: 39) |
| CD28 | CD28 | NG_029618.1 | ACCAAAATCTTGTTTCCTGG (SEQ ID NO: 40) |
| | | | CACCAAAATCTTGTTTCCTG (SEQ ID NO: 41) |
| CD4 | CD4 | NG_027688.1 | CTCAGGGAAAGAAAGTGGTG (SEQ ID NO: 42) |
| | | | CAGGGAAAGAAAGTGGTGCT (SEQ ID NO: 43) |
| | | | CAGGGAAAGAAAGTGGTGCT (SEQ ID NO: 44) |
| CD8 | CD8A | NG_011608.2 | CCGGAACTGGCTCGGCCTGG (SEQ ID NO: 45) |
| | | | CACCCGGAACTGGCTCGGCC (SEQ ID NO: 46) |
| | | | AGCCAGTTCCGGGTGTCGCC (SEQ ID NO: 47) |
| Interleukin 2 | IL2RA | NG_007403.1 | GGCAGGTAAGGGCCTGTGGG (SEQ ID NO: 48) |
| receptor | | | CCTGGCTGCCAGGCAGGTAA (SEQ ID NO: 49) |
| subunit alpha | | | |
| Interleukin 15 | ILI5RA | NC_000010.11 | GCCGCCGGCGACGCGGGGTA (SEQ ID NO: 50) |
| receptor | | | CTCCGGCCGCCGGCGACGCG (SEQ ID NO: 51) |
| subunit alpha | | | |
| Interleukin 21 | 1L21R | NG_012222.1 | CGGTGTAGCAGACGAGGTCG (SEQ ID NO: 52) |
| receptor | | | AGGTCGGGGCAGCCCCAGCC (SEQ ID NO: 53) |
| NKp46 | NCR1 | NC_000019.10 | AAGGAAGGACTCACGCTGCT (SEQ ID NO: 54) |
| | | | GAAGGAAGGACTCACGCTGC (SEQ ID NO: 55) |
| NKp44 | NCR2 | NC_000006.12 | AGGCTCTCAGGCACAATCCA (SEQ ID NO: 56) |
| | | | TCAGGCACAATCCAAGGCTC (SEQ ID NO: 57) |
| NKp30 | NCR3 | NG_021176.1 | CTGGGACACCCAGAGAGCAC (SEQ ID NO: 58) |
| | | | CCCAGGATCCTGTGCTCTCT (SEQ ID NO: 59) |
| NKG2D | KLRK1 | NG_027762.1 | ATACGTACATCTGCATGCAA (SEQ ID NO: 60) |
| | | | GTTCCTGGCTTTTATTGAGA (SEQ ID NO: 61) |
| | | | GACCAAACCGACTAGACAGG (SEQ ID NO: 62) |
| CD16 | FCGR3A | NG_009066.1 | AGGGACCAAACCGACTAGAC (SEQ ID NO: 63) |
| | | | TTTACTTCCTCCTGTCTAGT (SEQ ID NO: 64) |
| 2B4 | CD244 | NG_015991.1 | TTCTCTTTCCTAGGATGCCA (SEQ ID NO: 65) |
| | | | GTTCTCTTTCCTAGGATGCC (SEQ ID NO: 66) |
| | | | CAGCTGATCCCTGGCATCCT (SEQ ID NO: 67) |
| CD94 | KLRD1 | NC_000012.12 | GCTCATTTAAATGTTTCTTG (SEQ ID NO: 68) |
| | | | GTGAAGATAAAAATCGTTAT (SEQ ID NO: 69) |
| CD2 | CD2 | NG_050908.1 | CAAGGCATTCGTAATCTCTT (SEQ ID NO: 70) |
| | | | CAAAGAGATTACGAATGCCT (SEQ ID NO: 71) |
| CD69 | CD69 | NC_000012.12 | ATGTTTCCTTATTATTTGTA (SEQ ID NO: 72) |
| | | | AACAAACCTTACAAATAATA (SEQ ID NO: 73) |
| | | | AATGTGAGAAGAATTTATAC (SEQ ID NO: 74) |
| CD27 | CD27 | NG_031995.1 | AGTAGCTGAGAGCCCCACCA (SEQ ID NO: 75) |
| | | | CCTCTCTGGGCAGCTCTTGG (SEQ ID NO: 76) |
| | | | TCTGGGCAGCTCTTGGGGGC (SEQ ID NO: 77) |
| | | | TCTGGGCAGCTCTTGGGGGC (SEQ ID NO: 78) |
| DNAM-1 | CD226 | NC_000018.10 | TATTTCAGCTCTATGTGAAG (SEQ ID NO: 79) |
| | | | CTCTATGTGAAGAGGTGCTT (SEQ ID NO: 80) |
| DAP10 | HCST | NC_000019.10 | GCTGCAGCTCAGACGACTCC (SEQ ID NO: 81) |
| | | | CGTCTGAGCTGCAGCCACTG (SEQ ID NO: 82) |
| DAP12 | TYROBP | NG_009304.1 | GACAGGACGGAGACCTGAGG (SEQ ID NO: 83) |
| | | | CTGGACAGGACGGAGACCTG (SEQ ID NO: 84) |
| | | | GGGCCTUGGCCTGGACAGGA (SEQ ID NO: 85) |
| FcRγ | FCER1G | NG_029043.1 | GCAGAGCTGAGGCTCTCCCA (SEQ ID NO: 86) |
| | | | TCTATCCCCTCAGCGGCCCT (SEQ ID NO: 87) |
| IL12RB1 | ILI2RB1 | NG_007366.2 | ACAGCACTCACTGGTTCTGC (SEQ ID NO: 88) |
| | | | GGTTCTGCAGGCAGCTGCAA (SEQ ID NO: 89) |
| | | | GTCCTGAAAACAGCACTCAC (SEQ ID NO: 90) |
| IL12RB2 | IL12RB2 | NG_032977.1 | CTGTTGATTAAAGCAAAAAT (SEQ ID NO: 91) |
| | | | ATTGCAGATGCGTGCAAGAG (SEQ ID NO: 92) |
| IL18RA | IL18R1 | NC_000002.12 | CCTTCAACCACAGTAATGTG (SEQ ID NO: 93) |
| | | | CTTATATCTGTAAGCACTGC (SEQ ID NO: 94) |

Regardless of the method used to introduce exogenous nucleic acids into a host cell, in order to confirm the presence of the recombinant DNA sequence in the host cell a variety of assays may be performed. Such assays include, for example, Southern and Northern blotting, RT-PCR and PCR, or by detecting the presence or absence of a particular protein or peptide, e.g., by immunological means (ELISAs and Western blots).

### Therapy

Herein described is also a method of treating a disease associated with expression of a target antigen. This should be understood to encompass reducing or otherwise ameliorating a disease in a mammal, i.e., a reduction or amelioration of any one or more symptoms of disease,

Although it is always most desirable to achieve the cure of a disease, there is nevertheless significant clinical value in slowing the progression of a disease. For example, in the context of a viral infection such as HIV or HBV even if complete cure cannot be achieved, a reduction in the extent of viral load and spread may provide a means of controlling the infection such that the severe immunodeficiency of HIV, for example, which is ultimately fatal is not experienced and a relatively normal life span can be achieved without the severe side effects that are characteristic of the current anti-viral drug cocktails which patients are required to take. In the specific context of neoplastic conditions, modified immune cells disclosed herein, such as modified T cells, when administered to a patient, down-regulate the growth of a neoplasm. Reference to “growth” of a cell or neoplasm should be understood as a reference to the proliferation, differentiation and/or maintenance of viability of the subject cell, while “down-regulating the growth” of a cell or neoplasm is a reference to the process of cellular senescence or to reducing, preventing or inhibiting the proliferation, differentiation and/or maintenance of viability of the subject cell. In a preferred embodiment the subject growth is proliferation and the subject down-regulation is CD8+ T cell mediated killing. In this regard, the killing may be evidenced either by a reduction in the size of the tumor mass or by the inhibition of further growth of the tumor or by a slowing in the growth of the tumor. In this regard, and without limiting the present invention to any one theory or mode of action, the neoplastic cells may be killed by any suitable mechanism such as direct lysis or apoptosis induction or some other mechanism which can be facilitated by CD4+ or CD+ T cells, or T cells lacking these CD4 and CD8 markers. The present invention should therefore be understood to encompass reducing or otherwise ameliorating a neoplastic condition in a mammal. This should be understood as a reference to the prevention, reduction or amelioration of any one or more symptoms of a neoplastic condition. Symptoms can include, but are not limited to, pain at the site of tumor growth or impaired metabolic or physiological bodily functions due to the neoplastic condition. It should be understood that the method of the present invention may either reduce the severity of any one or more symptoms or eliminate the existence of any one or more symptoms. The method of the present invention also extends to preventing the onset of any one or more symptoms.

Accordingly, the method of the present invention is useful both in terms of therapy and palliation. To this end, reference to “treatment” should be understood to encompass both therapy and palliative care. As would be understood by the person of skill in the art, although it is always the most desirable outcome that a neoplastic condition is cured, there is nevertheless significant benefit in being able to slow down or halt the progression of the neoplasm, even if it is not fully cured. Without limiting the present invention in any way, there are some neoplastic conditions which, provided they are sufficiently down-regulated in terms of cell division, will not be fatal to a patient and with which the patient can still have a reasonable quality of life. Still further, it should be understood that the present method provides a useful alternative to existing treatment regimes. For example, in some situations the therapeutic outcome of the present method may be equivalent to chemotherapy or radiation but the benefit to the patient is a treatment regime which induces either fewer side effects or a shortened period of side effects and will therefore be better tolerated by the patient. As detailed above, it should also be understood that the term “treatment” does not necessarily imply that a subject is treated until total recovery. Accordingly, as detailed above, treatment includes reducing the severity of an existing condition or amelioration of the symptoms of a particular condition or palliation. In this regard, where the treatment of the present invention is applied at the time that a primary tumor is being treated it may effectively function as a prophylactic to prevent the onset of metastatic cancer. For example, for certain types of solid tumors, it may still be most desirable to surgically excise the tumor. However, there is always a risk that the entirety of the tumor may not be successfully removed or that there may be escape of some neoplastic cells. In this case, by applying the method of the present invention to lyse any such neoplastic cells, the method is effectively being applied as a prophylactic to prevent metastatic spread.

In accordance with this aspect of the invention, the subject cells are preferably autologous cells which are isolated and genetically modified ex vivo and transplanted back into the individual from which they were originally harvested. However, it should be understood that the present invention nevertheless extends to the use of cells derived from any other suitable source where the subject cells exhibit a similar histocompatability profile as the individual who is the subject of treatment, so that the transferred cells can perform their function of removing unwanted cells, before being subjected to immune rejection by the host. Accordingly, such cells are effectively autologous in that they would not result in the histocompatability problems which are normally associated with the transplanting of cells exhibiting a foreign MHC profile. Such cells should be understood as falling within the definition of being histocompatible. The cells may also have been engineered to exhibit the desired major histocompatability profile. The use of such cells overcomes the difficulties which are inherently encountered in the context of tissue and organ transplants.

However, where it is not possible or feasible to isolate or generate autologous or histocompatible cells, it may be necessary to utilise allogeneic cells. “Allogeneic” cells are those which are isolated from the same species as the subject being treated but which exhibit a different MHC profile. Although the use of such cells in the context of therapeutics could result in graft vs host problems, or graft rejection by the host, this problem can nevertheless be minimised by use of cells which exhibit an MHC profile exhibiting similarity to that of the subject being treated, such as a cell population which has been isolated/generated from a relative such as a sibling, parent or child or which has otherwise been generated in accordance with the methods exemplified herein.

It would be appreciated that in a preferred embodiment the cells which are used are autologous. However, due to the circumstances of a given situation, it may not always be possible to access an autologous cell population. In this case, and as detailed hereinbefore, it may be desirable or necessary to use syngeneic or allogeneic cells, such as cells which have been previously transfected and are available as frozen stock in a cell bank. Such cells, although allogeneic, may have been selected for transformation based on the expression of an MHC haplotype which exhibits less immunogenicity than some haplotypes which are known to be highly immunogenic or which has otherwise been generated in accordance with the methods exemplified herein.

Reference to an “effective number” means that number of cells necessary to at least partly attain the desired effect, or to delay the onset of, inhibit the progression of, or halt altogether the onset or progression of the particular condition being treated. Such amounts will depend, of course, on the particular condition being treated, the severity of the condition and individual patient parameters including age, physical conditions, size, weight, physiological status, concurrent treatment, medical history and parameters related to the disorder in issue. One skilled in the art would be able to determine the number of cells of the present invention that would constitute an effective dose, and the optimal mode of administration thereof without undue experimentation, this latter issue being further discussed hereinafter. These factors are well known to those of ordinary skill in the art and can be addressed with no more than routine experimentation. It is preferred generally that a maximal cell number be used, that is, the highest safe number according to sound medical judgement. It will be understood by those of ordinary skill in the art, however, that a lower cell number may be administered for medical reasons, psychological reasons or for any other reasons.

As hereinbefore discussed, it should also be understood that although the method of the present invention is predicated on the introduction of genetically modified cells to an individual suffering a condition as herein defined, it may not necessarily be the case that every cell of the population introduced to the individual will have acquired or will maintain the subject modification. For example, where a transfected and expanded cell population is administered in total (i.e. the successfully modified cells are not enriched for), there may exist a proportion of cells which have not acquired or retained the genetic modification. The present invention is therefore achieved provided that the relevant portion of the cells introduced constitute the “effective number” as defined above.

The cells which are administered to the patient can be administered as single or multiple doses by any suitable route. Preferably, and where possible, a single administration is utilised. Administration via injection can be directed to various regions of a tissue or organ, depending on the type of treatment required.

In accordance with the method of the present invention, other proteinaceous or non-proteinaceous molecules may be co-administered with the introduction of the transfected cells. By “co-administered” is meant simultaneous administration in the same formulation or in different formulations via the same or different routes or sequential administration via the same or different routes. By “sequential” administration is meant a time difference of from seconds, minutes, hours or days between the transplantation of these cells and the administration of the proteinaceous or non-proteinaceous molecules. For example, depending on the nature of the condition being treated, it may be necessary to maintain the patient on a course of medication to alleviate the symptoms of the condition until such time as the transplanted cells become integrated and fully functional (for example, the administration of anti-viral drugs in the case of an HIV patient). Alternatively, at the time that the condition is treated, it may be necessary to commence the long term use of medication to prevent re-occurrence of the condition. For example, where the subject damage was caused by an autoimmune condition, the ongoing use of a low level of immunosuppressive drugs may be required once the autoreactive cells have been destroyed.

It should also be understood that the method of the present invention can either be performed in isolation to treat the condition in issue or it can be performed together with one or more additional techniques designed to facilitate or augment the subject treatment. These additional techniques may take the form of the co-administration of other proteinaceous or non-proteinaceous molecules or surgery, as detailed hereinbefore.

Yet another aspect of the present invention is directed to the use of T cells or NK cells genetically modified, as hereinbefore defined in the manufacture of a medicament for the treatment of a condition characterised by the presence of an unwanted population of cells in a mammal.

The present description is further illustrated by the following examples, which should not be construed as limiting in any way. The contents of all cited references (including literature references, issued patents and published patents applications, as cited throughout this application) are hereby expressly incorporated by reference.

### EXAMPLES

Example 1—Generation of T Cells Expressing Anti-TAG-72 scFv FP Using CRISPR/Cas9 Knock-In to CD3 Gene

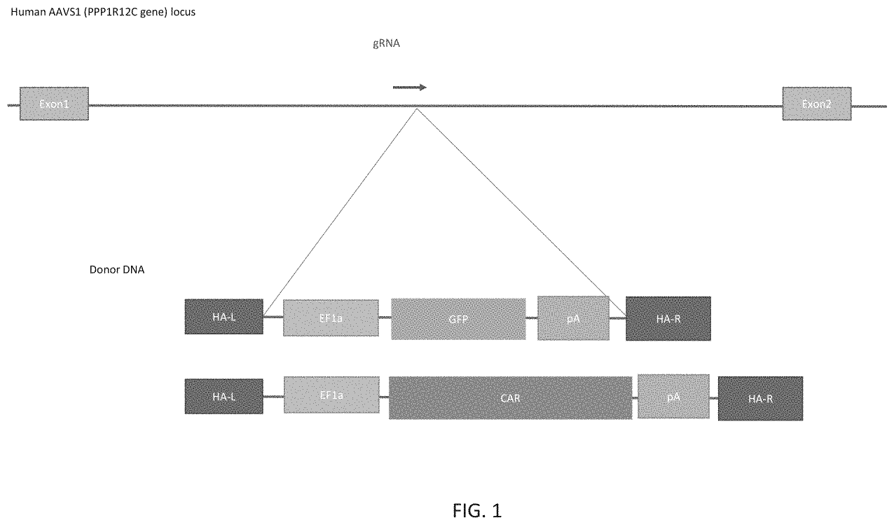

CAR transgene expression via lentiviral or retroviral random integration into the genome usually is subjected to position effects and silencing. In addition, random gene insertion might interrupt or activate the neighbouring genes. Genomic safe harbor sites are transcriptionally active, therefore allowing robust and stable gene expression. Furthermore, a transgene insertion at genomic safe harbor sites does not have adverse effects on the host cell genome. For human cells, AAVS1 has been accepted as a high gene expression and a safe harbor site in human genome (Oceguera-Yanez et al., Methods, 2016. 101: p. 43-55). CRISPR, TALEN or ZFN technologies can be utilized to target gene insertion at these genomic loci. In order to generate a viral-free and site-specific integrated CAR-T cell, we used the CRISPR/Cas9 technology to introduce the transgene into a specific site, AAVS1 (FIG. 1).

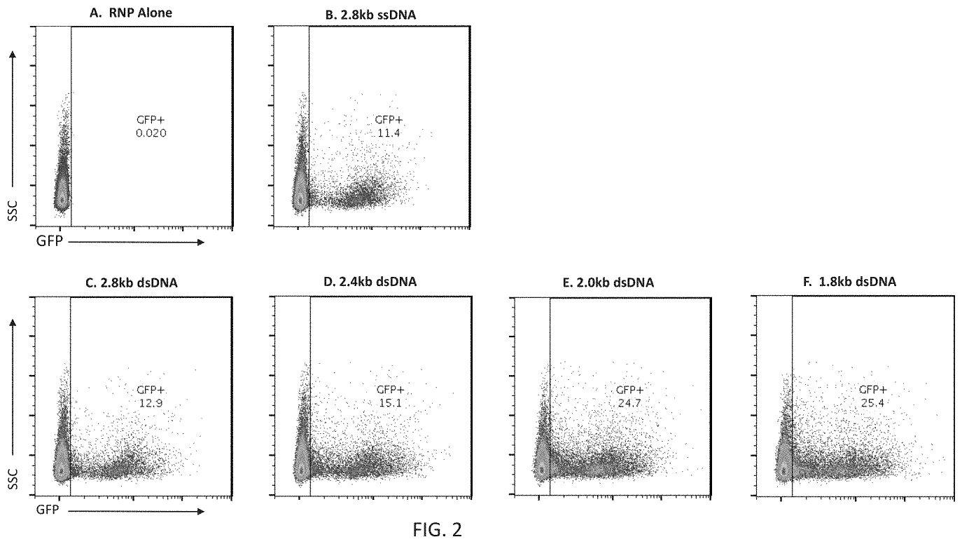

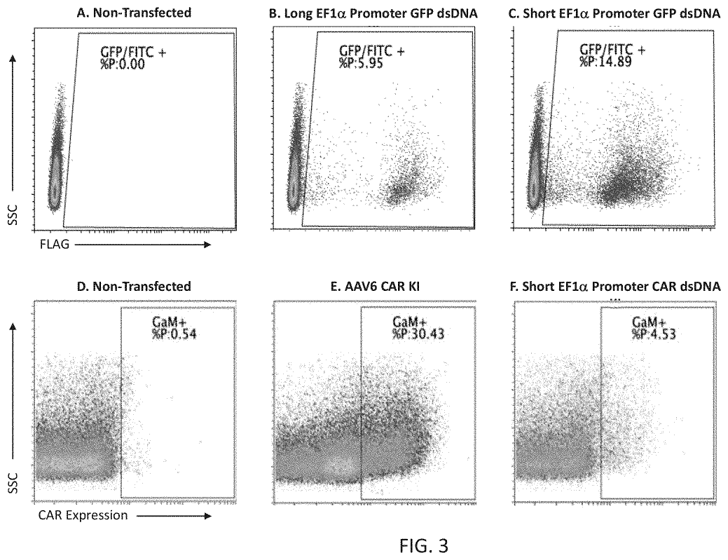

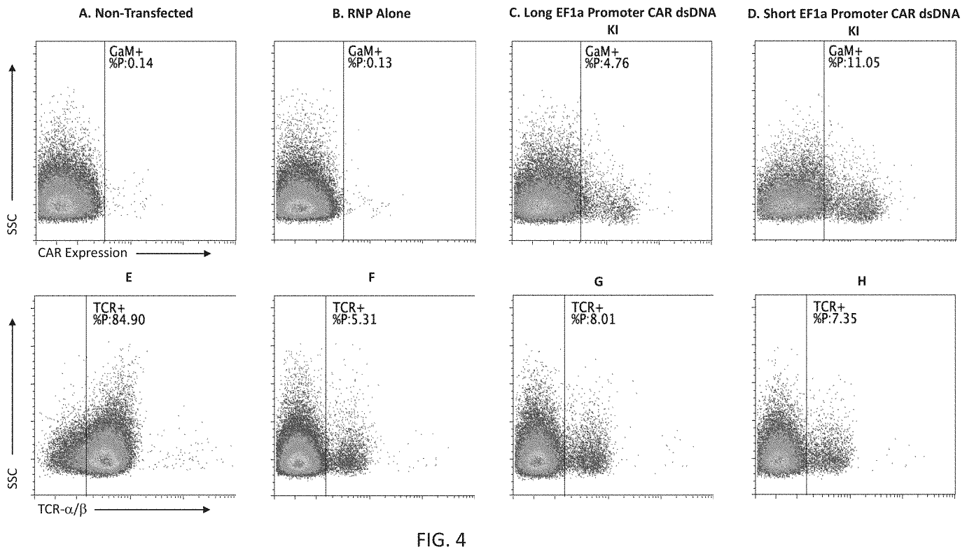

In order to knock-in (KI) GFP and CAR gene expression cassettes into the AAVS1 locus without using a virus vector, we used CRISPR guide RNA [SEQ ID NO: 7] targeting AAVS1 locus and GFP or CAR donor DNA for T cell electroporation (FIG. 1). ssDNA [SEQ ID NO: 13] and dsDNA donor DNA [SEQ ID NOS: 13, 14, 15 and 16] can both be inserted into AAVS1 locus after co-transfection with AAVS1 RNP into human activated T cells, and GFP could be stably expressed in T cells. Using the GFP expression cassette, we found the size or length of donor DNA dramatically reduced the non-viral genome KI efficiency (FIG. 2). An ˜33% reduction in DNA length (from 2.8 kb to 1.8 kb) resulted in a greater than 100% increase in KI efficiency. By reducing the length of homologous arms (FIG. 2) or promoter (FIGS. 3 and 4), we were able to increase the GFP or CAR non-viral genome KI efficiency into the AAVS1 or TRAC locus (as compared to that reported by Eyquem et al., Nature, 2017, 543(7643): p. 113-117).

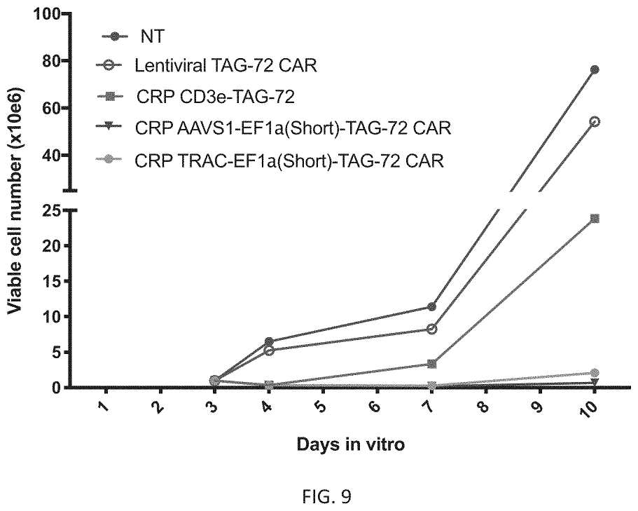

In contrast to GFP, the size of a second-generation CAR coding sequence is usually around 1.5 kb long; nearly double the size of the GFP gene, and the entire CAR gene expression cassette with homological arms, promoter, coding sequence and polyA sequence is larger than 3 kb (FIG. 1). Unsurprisingly, previous attempts to use non-viral insertion of CAR genes into the AAVS1 locus have resulted in poor KI efficiency (4.53%) compared with use of Adeno-Associated Virus 6 (AAV6) (30,43%) as a vector for donor DNA delivery (FIG. 3) (Wang et al., Nucleic Acids Res, 2016. 44(3): p. e30). In addition, non-viral insertion of CAR genes into the AAVS1 or TRAC locus resulted in poor cell viability and impaired the in vitro expansion and growth of T cells (FIG. 9). These results indicate that a CAR gene expression cassette is too large for non-viral transfection of T cells for immunotherapy.

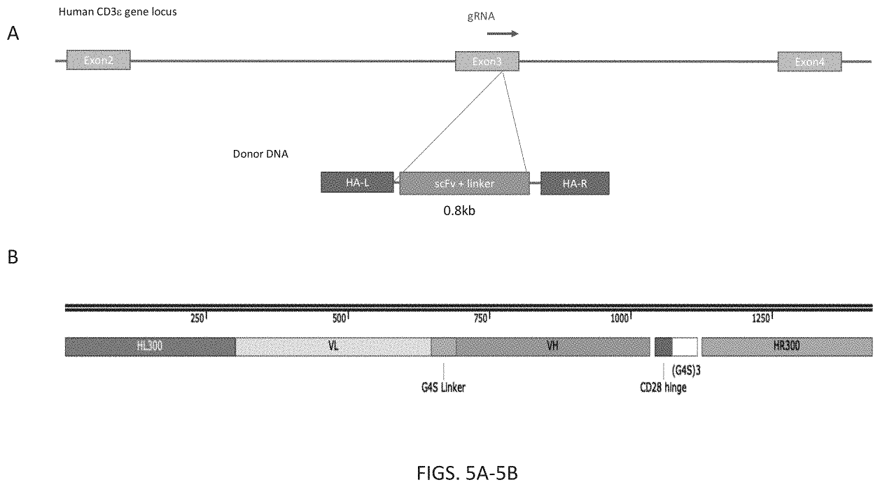

The scFv is the crucial sequence for tumor antigen binding, the size of which is around 0.8 kb (with a linker), about half the size of a CAR gene and therefore potentially amenable to non-viral delivery and knock-in to the T cell genome. Utilising the strategy shown in FIG. 5, we used the CRISPR/Cas9 system to KI a TAG-72 scFv and a linker into the N-terminus of CD3 to link the tumor antigen binder with the T cell receptor complex, and enable T cells to recognise TAG-72 positive tumor cells. An advantage of this approach is that it utilizes the endogenous cis-regulatory elements of CD3 and doesn't require inclusion of artificial promoter and poly A terminator sequences for transgene expression (FIG. 5).

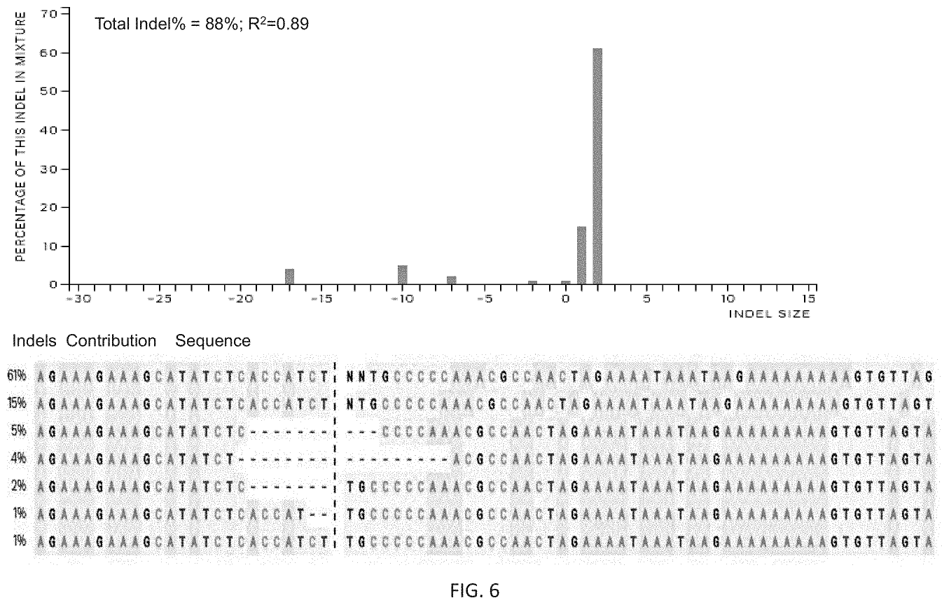

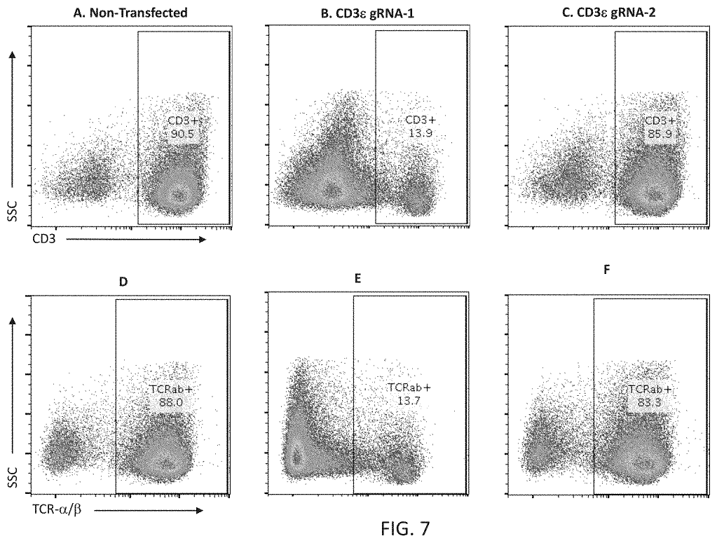

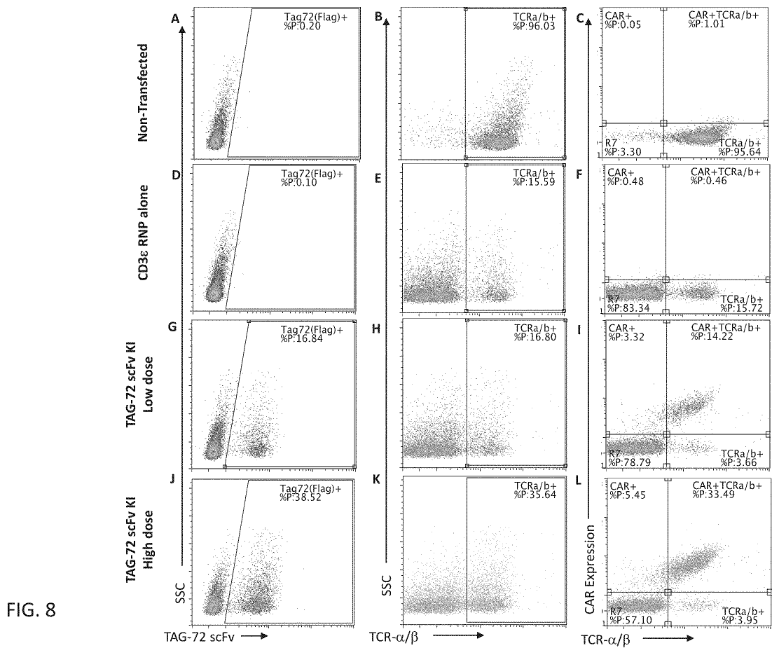

To produce the CD3ϵ FP expressing cells, we firstly verified two CRISPR guide RNAs (gRNA-1 [SEQ ID NO: 1] and gRNA-2 [SEQ ID NO: 2]) targeting CD3ϵ, N-terminus to knock-in the scFv and linker sequence just after the CD3ϵ signal peptide sequence. CD3ϵ gRNA-1 [SEQ ID NO: 1] showed high activity to introduce indels into CD3ϵ N-terminus (FIG. 6) and higher knock-out efficiency of CD3 protein in T cells (FIG. 7). To further create the CRISPR anti-TAG-72/CD3ϵ FP T cells, two doses of anti-TAG-72 scFv donor DNA [SEQ ID NO: 8 or 9] were knocked-into the CD3ϵ locus after co-transfection with CD3ϵ gRNA-1 RNP. By increasing the donor DNA dose, we could achieve 38.5% KI efficiency (FIG. 8), which is much higher than the minimum transduction efficiency (5%) required for lentiviral or retroviral CAR-T cell therapy (Porter et al., N Engl J Med, 2011. 365(8): p. 725-33; Brentjens et al., Sci Transl Med, 2013. 5(177): p. 177ra38). As compared with the CD3ϵ KO cells, which lost most of the TCRα/β complex on the cell surface, CRISPR anti-TAG-72/CD3ϵ FP T cells rescued the formation of TCRα/β complex on the cell surface in a dose dependent manner (FIG. 8). This result indicates that the anti-TAG-72 scFv peptide was fused to the N-terminus of the endogenous CD3ϵ protein, and the anti-TAG-72-CD3ϵ FP successfully replaced the endogenous CD3ϵ to incorporate into the natural TCRα/β0 complex. Moreover, the CRISPR anti-TAG-72/CD3ϵ FP T cells could be expanded as well as lentiviral anti-TAG-72 CAR-T cells in vitro for cell therapy applications (FIG. 9).

### Human T-Cell Isolation and Culture

Primary human T cells were isolated from healthy human donors either from fresh whole blood, or from huffy coats obtained from the Australian Red Cross Blood Service (non-conforming/discarded material not suitable for clinical purposes). All patients and healthy donors provided informed consent. Peripheral blood mononuclear cells (PBMCs) were isolated by Ficoll-Paque (GE Healthcare, Illinois, United States) centrifugation using Leucosep™ tubes (Greiner, Kremsmünster, Austria) as per manufactures instructions. PBMCs were cryopreserved prior to use. For use in transfections, PBMCs were thawed and T-cells were isolated and activated using Dynabeads® Human T-Activator CD3/CD28 beads (Thermo Fisher, Massachusetts, United States). Cells and beads were incubated at 1:3 ratio for 1 hour at room temperature, with continual gentle mixing. Unbound cells were then removed by placing cell-bead suspension on a magnet for 1-2 mins. The supernatant was removed and cell-bead mixture was incubated for ˜65 hrs at 37° C. 5% CO₂ in T-cell medium: TexMACS Medium (Miltenyl Biotech, Bergisch Gladbach, Germany) with 5% human AB serum (Sigma-Aldrich, Missouri, United States) and 100 U/mL IL-2. T-cells were collected by dissociation of the cell-bead complexes by mixing 20-50×, immediately placed on a magnet for 1-2 mins and the cell containing supernatant collected. The isolated T-cell suspension was counted on a MUSE cell counter (Merck-Millipore, Massachusetts, United States) and prepared for transfection.

### Primary Human T Cell Culture and Transection

For Cas9 RNP transfections, the human CD3+CD28+ T cells were isolated as described above. Cas9 RNPs were prepared before transfection by incubating Cas9 protein with the chemical-modified synthetic guide RNAs at 1:2 ratio at room temperature for 15 minutes. Chemically synthesized Modified sgRNA (Synthego) or crRNAs and tracrRNA (Synthego, IDT) annealed guide RNAs were both tested, dsDNA was amplified using high-fidelity tag polymerase (New England Biolabs, Massachusetts, United States) and purified by PCR purification kit (Qiagen, Hilden, Germany). ssDNA was produced by Guide-it Long ssDNA Production System (Takara Bio Inc, Shiga Prefecture, Japan). To transfect the Cas9 RNP and DNA, T cells were electroporated with a Neon transfection system (Thermo Fisher) or 4D-Nucleofector System (Lonza, Basel, Switzerland). As a control, lentiviral CAR vectors were used to transduce the activated human CD3+ T cells. To produce the lentiviral CAR-T cells, the activated human CD3+CD28+ T cells were incubated with the lentiviral particles in Retronectin (Takara Bio Inc) coated plates for 48 hours at a multiplicity of infection (MOI) of 50.

### Flow Cytometry and Cell Sorting

To detect the surface expression of scFv-CD3 or CAR constructs, flow cytometric analysis was performed on a MACSQuant® Analyzer 10 (Miltenyi Biotec, Bergisch Gladbach, Germany) or cell sorting using the BD Aria (BD Biosciences). For cell surface staining, for either flow cytometry or cell sorting, cells were collected by centrifugation and resuspended at 10 uL/1×10e6 cells in the appropriate antibody cocktail in FACS buffer [3% human serum albumin (HSA, CSL, Melbourne, Australia) in phosphate buffered saline, in particular dPBS (Sigma-Aldrich, Missouri, United States)]. Cells were incubated for 15 mins at 4° C. in the dark and washed once in FACS buffer before resuspension in appropriate solution for flow cytometric analysis or cell sorting. Depending on the construct, either goat anti-mouse IgG F(ab′)2 antibody (Jackson ImmunoResearch Laboratories Inc, Philadelphia, United States) or anti-Flag-M2 antibody (Sigma-Aldrich). Propidiuin iodide solution (Miltenyi Biotec) or Viobility 405/520 dye were used to discriminate the live/dead cells.

### Quantitative Assessment of Genome Editing

The efficacy and the mutation spectrum of Cas9-CRISPR genome editing efficiency was analysed by Inference of CRISPR Edits (ICE) assay. Genomic DNA was extracted from cells 4 days after electroporation using ISOLATE II Genomic DNA Kit (Bioline) following manufacturer's instructions. PCR amplicons spanning the gRNA genomic target sites were generated using the High-Fidelity taq ploymerase (New England Biolabs). For analysing genetic modification frequencies using ICE, the purified PCR products were Sanger-sequenced and the sequence chromatogram was analyzed with the ICE software available on line.

### Example 2—In Vitro Function of Anti-TAG-72/CD3ϵ CRISPR FP T Cells

T cells expressing the anti-TAG-72-CD3ϵ FP construct, which we referred to as anti-TAG-72/CD3ϵ CRISPR FP T cells, were generated according to the methods described in Example 1. For comparative purposes, T cells expressing a TAG-72 CAR, as previously described (PCT/AU2016/051141 by Cartherics Pty. Ltd., published as WO 2017/088012), were generated using lentiviral transduction of the 2nd generation 4-1BBzeta CAR construct, using established methods (e.g., WO 2017/088012 by Cartherics Pty. Ltd.). Growth curves for the T cells are shown in FIG. 9. As compared to the CAR gene CRISPR KI T cells, which were barely expandable in vitro, the in vitro expansion rate of anti-TAG-72/CD3ϵ CRISPR FP T cells was much higher than the CAR gene CRISPR KI T cells. These results indicated that anti-TAG-72/CD 3s CRISPR FP T cells could be expanded in vitro for immunotherapy.

### T Cell in Vitro Cytotoxicity Assay

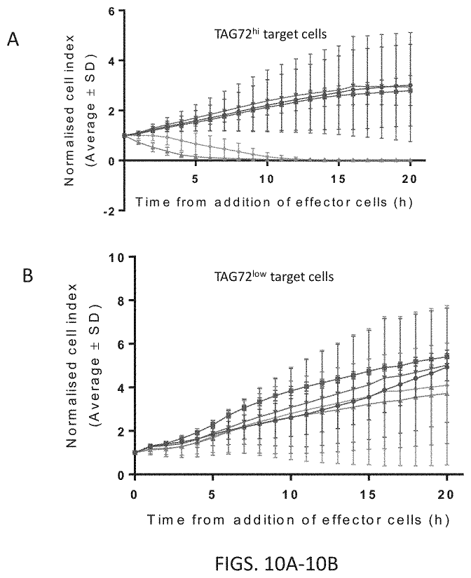

The real-time cell monitoring system (xCELLigence) was employed to determine the killing efficiency of FP T cells or CAR-T cells in vitro. 10,000 target cells/100 μL (for example the ovarian cancer cell line Ovcar3) were resuspended in culture media (for example, RPMI-1640 basal media) supplemented with 10%-20% fetal calf serum and bovine insulin and deposited into Real Time Cell Analysis microtitre ePlate compatible with the xCELLigence system. Target cells were maintained at 37° C., 5% CO₂ for 3-20 h to allow for cellular attachment. Following attachment of target cells, TAG-72 CAR-T effector cells or TAG-72 FPs T cells were added at various effector to target ratios ranging from 1:5 to 5:1. In some instances, effector cells were isolated based on GFP or Flag expression via FACS prior to use. In parallel, non-transfected T cells were co-cultured with target cells to demonstrate the background functionality of T cells in vitro. All co-cultures were maintained in optimal growth conditions for al least 20 h. Throughout, cellular impedance was monitored; a decrease in impedance is indicative of cell detachment and ultimately cell death. To compare the initial capacity of anti-TAG-72/CD3ϵ CRISPR FP T cells and lentiviral anti-TAG-72 CAR-T cells to lyse tumor cells, tumor cells with high or low levels of TAG-72 expression were incubated with anti-TAG-72/CD3ϵ CRISPR FP T cells, lentiviral CAR-T cells, or negative control effector T cells, and the in vitro cytotoxicities were monitored by xCELLigence. Anti-TAG-72/CD3ϵ CRISPR FP T cells killed TAG-72hi tumor cells as efficiently as anti-TAG-72 CAR-T cells, whereas no lysis of TAG-72low tumor cells (FIGS. 10A-10B) was observed.

### Example 3—Generation and In Vitro Activity of Anti-CD19/CD3ϵ CRISPR FP T Cells

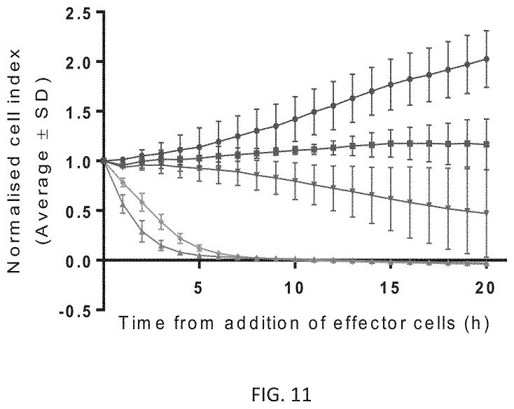

To demonstrate that the method for generating anti-TAG-72/CD3ϵ CRISPR FPs T cells is not limited to just tumor antigen TAG-72, equivalent anti-CD19 fusion proteins were generated. T cells expressing the anti-CD19/CD3ϵ FP construct, which we defined as anti-CD19/CD3ϵ CRISPR FP T cells, were generated according to the methods described in Example 1. To generate anti-CD19/CD3ϵ CRISPR FP T cells, anti-CD19 say donor DNA [SEQ ID NO: 10] was knocked-into CD3ϵ locus after co-transfection with CD3ϵ RNP gRNA-1 [SEQ ID NO: 1]. In vitro cytotoxicity of anti-CD19/CD3ϵ CRISPR FP T cells was compared with T cells expressing an anti-CD19 lentiviral CAR according to the methods described in Example 2. Anti-CD19/CD3ϵ CRISPR FP T cells killed CD19-hi tumor cells as efficiently as anti-CD19 CAR-T cells (FIG. 11). This result shows that our CD3ϵ CRIPSR FP method can be applied broadly to tumor antigens via knocking-in the relevant antibody scFv sequences into CD3ϵ N-terminus,

### Example 4—Generation of Anti-TAG-72 CD3δ and CD3γ CRISPR FP T Cells

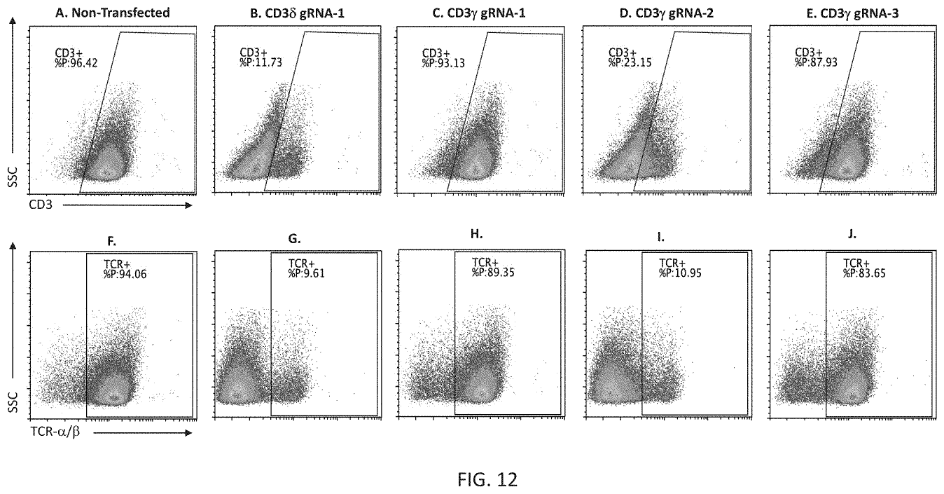

To demonstrate that the method for generating anti-TAG-72 FP T cells is not limited to just CD3ϵ, equivalent anti-TAG-72 fusion proteins were generated with CD36 and CD3γ. Anti-TAG-72/CD3δ and Anti-TAG-72/CD3γ CRISPR FP T cells were generated according to the methods described in Example 1. Firstly, the KO efficiency of CD3δ gRNA-1 [SEQ ID NO: 3] and CD3γ gRNAs (gRNA-1 [SEQ ID NO: 4], gRNA-2 [SEQ ID NO: 5], gRNA-3 [SEQ ID NO: 6]) to CD3δ or CD3γ N-terminus was assessed by FACS analysis of CD3 expression by T cells treated with the gRNAs. CD3δ gRNA-1 ([SEQ ID NO: 3]) and CD3γ gRNA-2 [SEQ ID NO: 5] showed the highest activity of specific indels introduction for CD3 knock-out, respectively (FIG. 12).

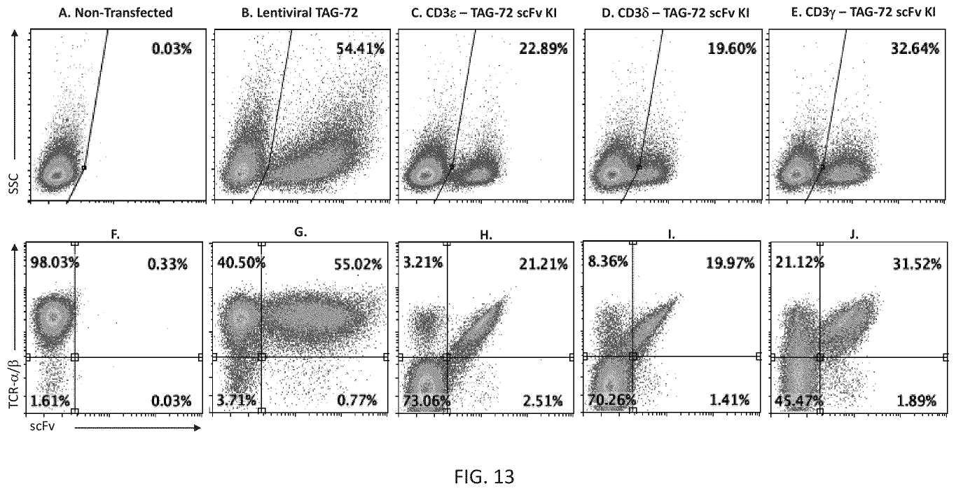

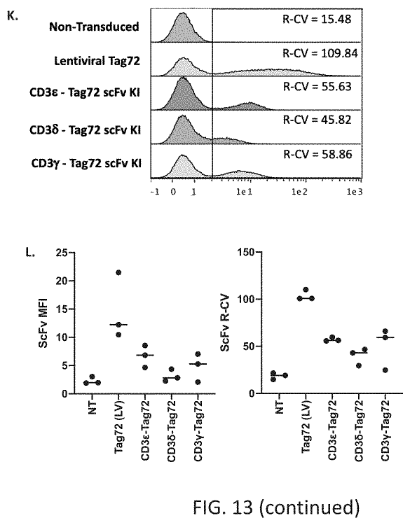

To further create the anti-TAG-72/CD3δ and anti-TAG-72/CD3γ CRISPR FP T cells, anti-TAG-72 say donor DNA [SEQ ID NO: 8 or 9] was knocked-into CD3δor the CD3γ locus Ether co-transfection with CD3δ gRNA-1 or CD3γ gRNA-2 RNP, respectively. FACS analysis of TAG-72 scFv and TCRα/β of anti-TAG-72 CD3δ or CD3γ CRISPR FP T cells shows that TAG-72 scFv donor DNA can be knocked-into CD3δ or CD3γ N-terminus and with retention of expression of the TCRα/β complex, equivalent to the CD3ϵ CRIPSR FP T cells of Examples 1 and 3 (FIG. 13A, Panels A to J), The CD3ϵ, CD3δ and CD3γ CRISPR FP cells had a lower but more homogenous transgene expression as compared with the viral transduced T cells without exposure to the tumor antigen (FIG. 13B, Panel K). We also found more homogenous and consistent transgene expression of CRISPR FP T cells in multiple donors as in contrast to the viral transduced T cells (FIG. 13B, Panel L), These results suggest that endogenous cis-regulatory elements are utilized for dynamic control of the transgene expression in CRISPR FP T cells, which can avoid cell over stimulation and exhaustion with antigen exposure.

### Example 5—In Vitro Function of Anti-TAG-72 CD3δ and CD3γ CRISPR FP T Cells

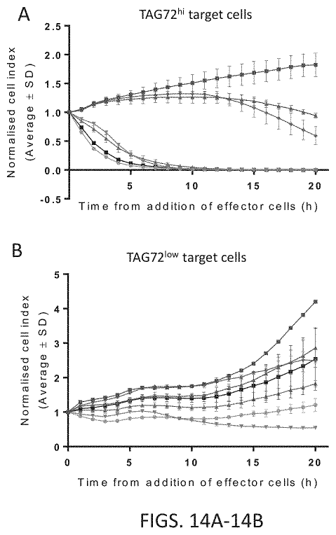

Anti-TAG-72 CD3δ and CD3γ0 CRISPR FP T cells were generated according to the methods described in Examples 1 and 4, and tested for their ability to kill tumor cells in vitro according to the methods described in Example 2. FIG. 14 shows the in vitro killing of TAG-72hi and TAG-72low target cells by anti-TAG-72 CD3ϵ, CD3δ and CD3γ CRISPR FP T cells. Not only CD3ϵ but also CD3δ and CD3γ CRISPR FP T cells show efficient killing of TAG-72hi tumor cells compared to anti-TAG-72 CAR-T cells, with less non-specific killing of TAG-72low tumor cells as compared with lentiviral CAR-T cells. In contrast to the lentiviral TFPs reported (PCT/US2016/033146 by TCR2 Therapeutics Inc., published as WO2016/187349), each of the CD3 FP T cells created using our CRISPR method was fully-functional in killing tumor cells in vitro. Lentivirally generated CD3δ, TCRα or TCRβ FP expressing T cells were not able to kill tumor cells in vitro (WO2016/187349). However, our CD 3δ FP T cells were able to kill tumor cells as efficiently as CD3ϵ and CD3γ FP T cells. This result indicates our method is more broadly applicable than the method of WO2016/187349.

### Example 6—In Vivo Function of Anti-TAG-72/CD3ϵ CRISPR FP T Cells

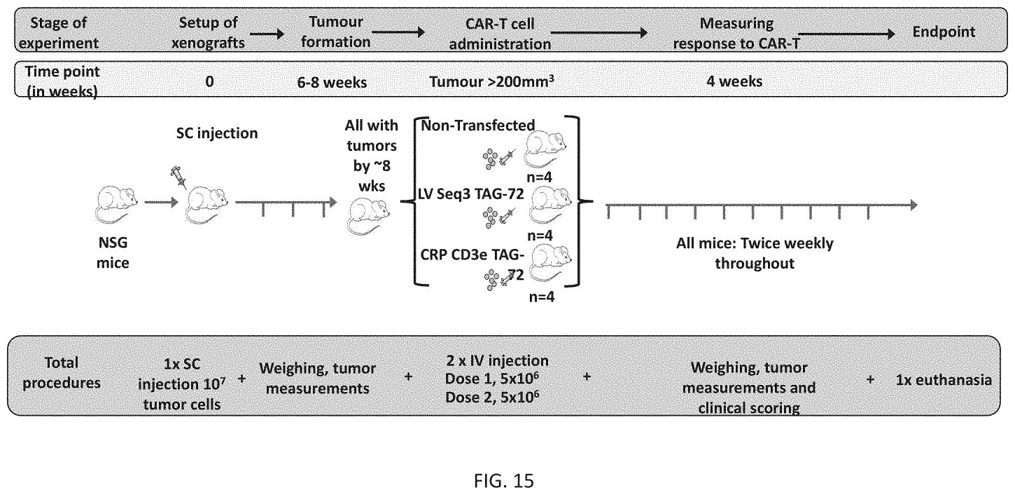

Anti-TAG-72/CD3ϵ CRISPR FP T cells were generated according to the methods described in Examples 1 and 2. For comparative purposes, T cells expressing an anti-TAG-72 CAR, as previously described (WO 2017/088012 by Catherics Pty. Ltd.), were generated using lentiviral transduction of the CAR construct using established methods. The T cells were assessed for their efficacy in an in vivo mouse solid tumor (xenograft) model (FIG. 15).

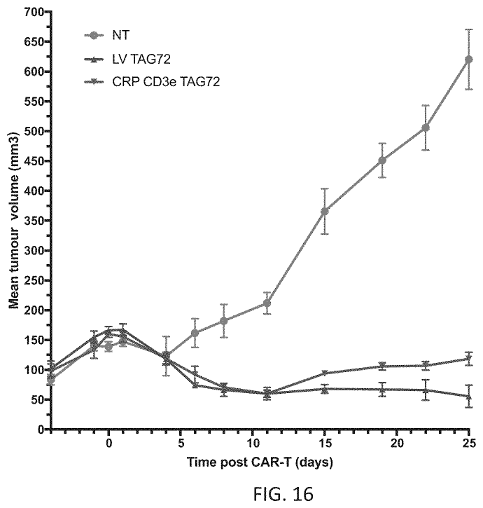

For this model, human tumors cell lines were grown on the flank of NSG mice by subcutaneously injecting approximately 1×10e7 human-derived TAG-72 positive Ovcar3 cancer cells into the flanks of 6-10 week old mice. Within 7 to 9 weeks, fully formed 200 mm³ tumors developed at the injection site. Once tumors reached this volume, the mice were randomized for treatment. CAR or FP T cell treatments were administered to the mice intravenously, with total of 2 injections of 5×10e6 T cells per injection. The results show that FP T cells are as efficient as CAR-T cells in their ability to kill tumor cells in vivo (FIG. 16).

### Example 7—In Vitro Function of CD3 Dual-Targeting CRISPR FP T Cells

To demonstrate that the method for producing CRISPR FP T cells can be developed to target multiple tumor antigens, anti-CD19/CD3ϵ+anti-TAG-72/CD3δ dual CRISPR FP T cells and anti-CD19/CD3ϵ anti-TAG-72/CD3γ dual CRISPR FP T cells were generated according to the methods described in Example 1.

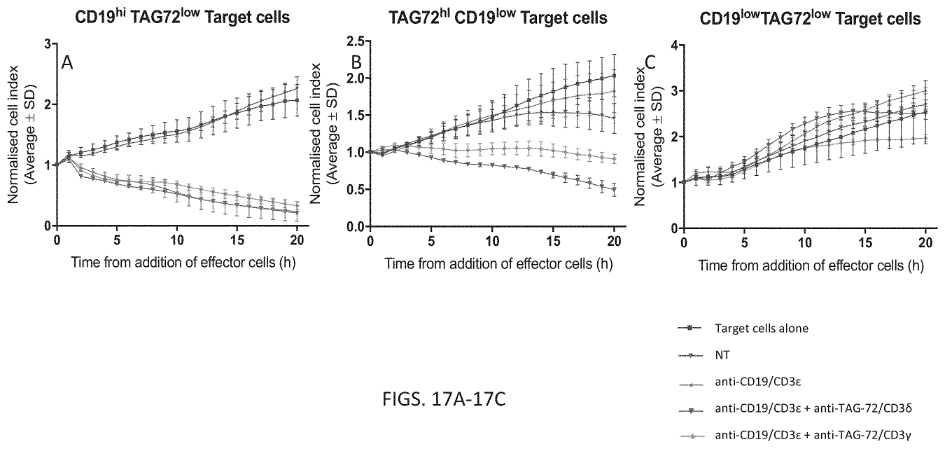

To generate anti-CD19/CD3ϵ+anti-TAG-72/CD3δ dual CRISPR FP T cells and anti-CD19/CD3ϵ+anti-TAG-72/CD3δ dual CRISPR FP T cells, anti-CD19 scFv donor DNA [SEQ ID NO: 10] with CD3ϵ RNP gRNA-1 [SEQ ID NO: 1] were co-transfected with anti-TAG-72 scFv donor DNA [SEQ ID NO: 8 or 9] and CD3δ gRNA-1 [SEQ ID NO: 3] or CD3γ gRNA-2 [SEQ ID NO: 5] RNP, respectively. In vitro cytotoxicity of anti-CD19/CD3ϵ+anti-TAG-72/CD3δ dual CRISPR FP T cells and anti-CD19/CD3ϵ+anti-TAG-72/CD3γ dual CRISPR FP T cells were compared with anti-CD19/CD3ϵ CRISPR FP T cells according to the methods described in Example 2. The anti-CD19/TAG-72 dual targeting CRISPR FP T cells killed CD19-hi TAG-72-low tumor cells as efficiently as CD19/CD3ϵ CRISPR FP T cells (FIG. 17). Moreover, anti-CD19/TAG-72 dual targeting CRISPR FP T cells could kill the CD19-low TAG-72-hi tumor cells efficiently while the CD19/CD3ϵ CRISPR FP T cells could not (FIG. 17). The killing mediated by anti-CD19/TAG-72 dual targeting CRISPR FP cells was also very specific which was evidenced by non-killing of CD19-low TAG-72-low tumor cells (FIG. 17). This result shows that our CRISPR FP method can be applied for multiple tumor antigens targeting at once via knocking-In the relevant antigen binding moiety sequences into different cell receptors at the same time.

### Example 8—In Vitro Function of CD3/CD28 Dual-Targeting CRISPR FP T Cells

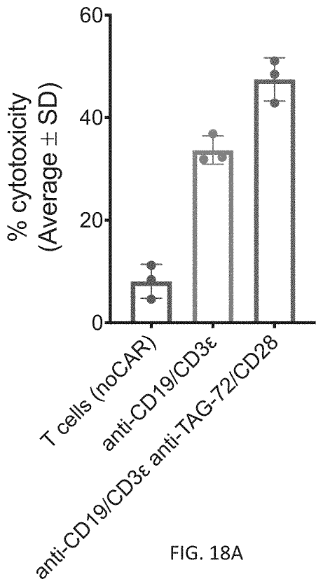

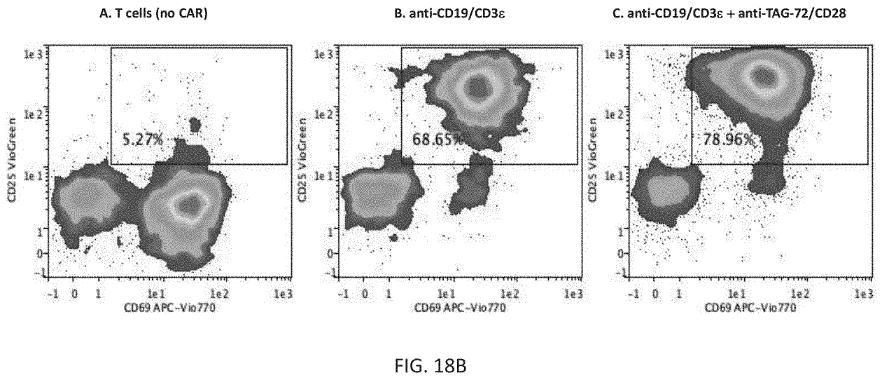

To demonstrate that the present invention can be used to produce dual targeting CRISPR FP T cells wherein the cell receptor is not a TCR, anti-CD19/CD3ϵ+anti-TAG-72/CD28 dual CRISPR FP T cells were generated according to the methods described in Example 1. To generate anti-CD19/CD3ϵ+anti-TAG-72/CD3δ dual CRISPR FP T cells, anti-CD19 scFv donor DNA [SEQ ID NO: 10] with CDR RNP gRNA-1 [SEQ ID NO: 1] RNP were co-transfected with anti-TAG-72 scFv donor DNA [SEQ ID NO: 96] and CD28 gRNA [SEQ ID NO: 40] RNP, respectively. Knock-in positive cells were isolated by fluorescence activated cell sorting (FACS) where, in brief, cells were incubated with either anti-Flag (to detect CD19 knock-in) or anti-F(ab′)2 (to detect TAG-72 knock-in) or both antibodies for 15 min at 4° C., protected from light. Cells were washed once before resuspending if FACS buffer. Single positive and double positive cells were isolated using the BD FACS Aria. Following isolation, cells were allowed to recover under normal growth conditions for at least 3 days before subsequent use. The ovarian cancer cell line, Ovcar3, was genetically modified by lentiviral transduction to generate a stable cell line overexpressing CD19 that was positive for both target antigens of interest—TAG-72 and CD19. This cell line is referred to herein as Ovcar3 (CD19). These cells were irradiated (30 Gy) before use in continued antigen exposure assay. Irradiated Ovcar3 (CD19) cells were seeded and allowed to develop monolayers before addition of CRISPR FP T cells. CRISPR FP T cells were moved to fresh irradiated Ovcar3 (CD19) monolayers daily for 7 days with complete media changes performed every alternate day. Following 7 days of continued antigen exposure, in vitro cytotoxicity of anti-CD19/CD3ϵ+anti-TAG-72/CD28 dual CRISPR FP T cells were compared with anti-CD19/CD3ϵ CRISPR FP T cells according to the methods described in Example 2. CAR-T function was calculated as % cytotoxicity=((Normalized CI_{target cells alone}−Normalized CIₜₑₛₜ)÷Normalized CI_{target cells alone})×100. Following continued antigen exposure, the anti-CD19/TAG-72 dual targeting CRISPR FP T cells demonstrated greater cytotoxicity than anti-CD19/CD3ϵ. CRISPR FP T cells (FIG. 18A). This result shows that our CRIPSR FP method can be applied for multiple tumor antigens targeting at once via knocking-in the relevant antigen binding moiety sequences into different cell receptors at the same time.

To demonstrate that T cell activation was enhanced through signaling of both CD3ϵ and CD28, CRISPR FT T cells that had been exposed to antigen expressing target cells (as described above) were characterised for the co-expression of activation markers CD25 and CD69 by flow cytometry (Eyquem et at, Nat Med (2019) 25(1): p. 82-88). Following continued antigen exposure, anti-CD19/TAG-72 dual targeting CRISPR FP cells demonstrated activation equivalent to the activation seen in the single targeting CRISPR FP T cells (FIG. 18B) highlighting the role of dual signaling in T cell activation and function.

### Example 9—Generation of Anti-TAG-72 2B4 FP NK Cells

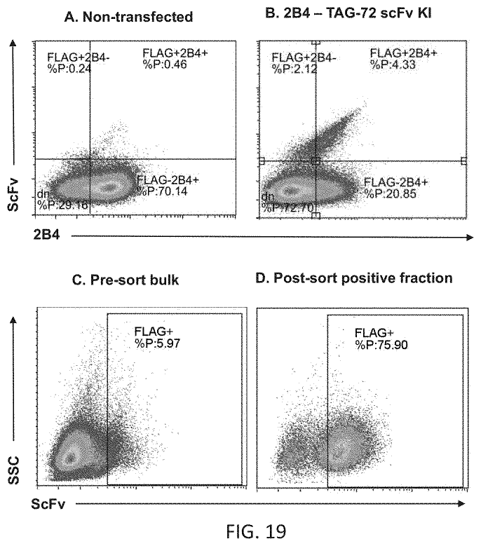

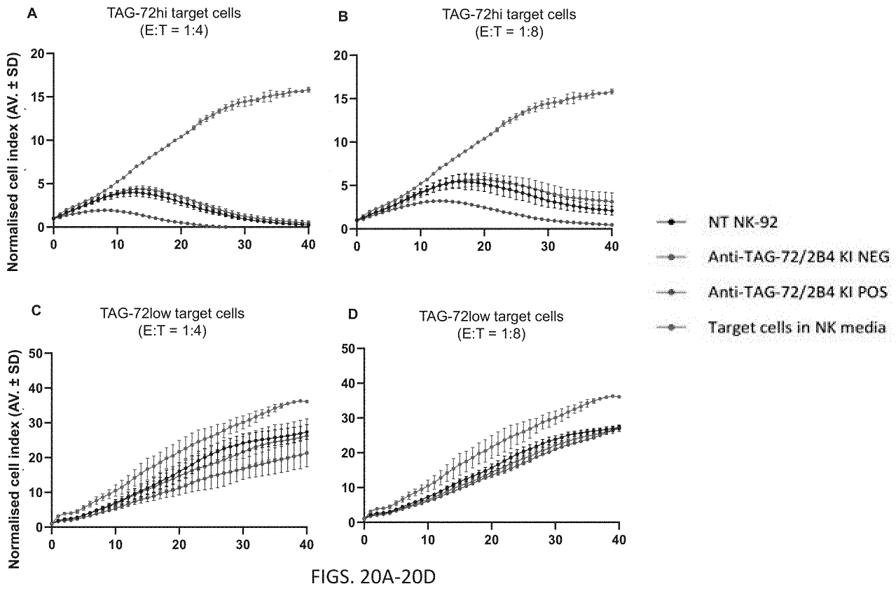

The receptor 2B4, also known as CD244, is a lymphocyte activation receptor highly expressed in NK cells and in certain populations of T cells. Ligation of 2B4 with specific antibody or ligand provides activating signal for NK cells (Waggoner et al., Front Immunol 3: 377 (2012)). To demonstrate that the method fur generating anti-TAG-72 CRISPR FP immune cells is not limited to just T cells, equivalent anti-TAG-72 CRISPR FP were generated with 2B4. Anti-TAG-72/2B4 CRISPR FP NK-92 cells were generated similarly to the methods described tar T cells (Example 1). To create the anti-TAG-72/2B4 CRISPR FP NK-92 cells, anti-TAG-72 scFv donor DNA with Flag tag [SEQ ID NO: 95] was knocked-into 2B4 gene (at the N terminus of 2B4 receptor) after co-transfection with 2B4 gRNA-3 [SEQ ID NO: 67] RNP. FACS analysis of TAG-72 scFv and 2B4 of transfected NK-92 cells shows that TAG-72 scFv donor DNA can be knocked-into the 2B4 N-terminus and with retention of expression of 2B4 receptor, equivalent to the CD3ϵ CRIPSR FP T cells of Examples 1 and 3 (FIG. 19B). In order to test their ability to kill tumor cells in vitro, anti-TAG-72/2B4 CRISPR FP NK-92 cells were purified by FACS isolation of Flag positive cells (FIG. 19 C-D), and then the positive fraction, negative fraction and non-transfected NK-92 cells were incubated with tumor target cells at different effector to target ratios (E:T) for xCELLigence assay according to the method described in Example 2. These results show that anti-TAG-72/2B4 CRISPR FP NK-92 cells can be created using the methodology developed for T cells as described in Example 1 to 5, and anti-TAG-72/2B4 CRISPR FP NK-92 cells can kill the TAG72 hi tumor cells specifically, and more efficiently than, the anti-TAG-72 scFv negative and non-transfected NK-92 cells (FIGS. 20A-D).

### Example 10—Generation of Say and Receptor CRISPR Knock-In FP iPSCs as a Cell Source for Adoptive Cell Therapy

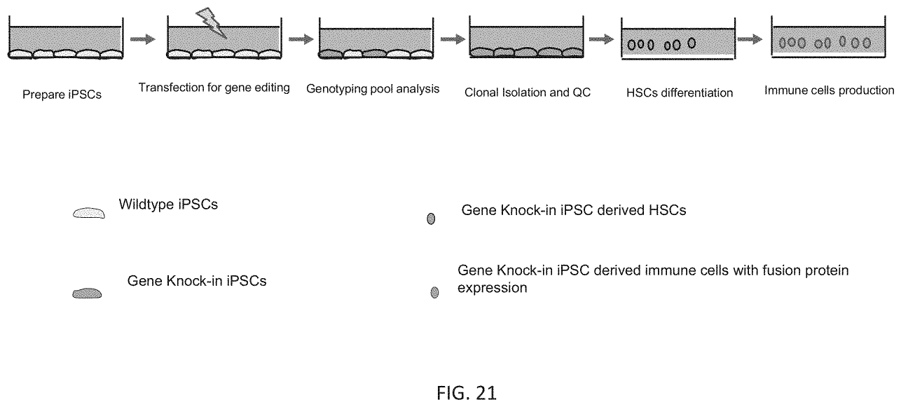

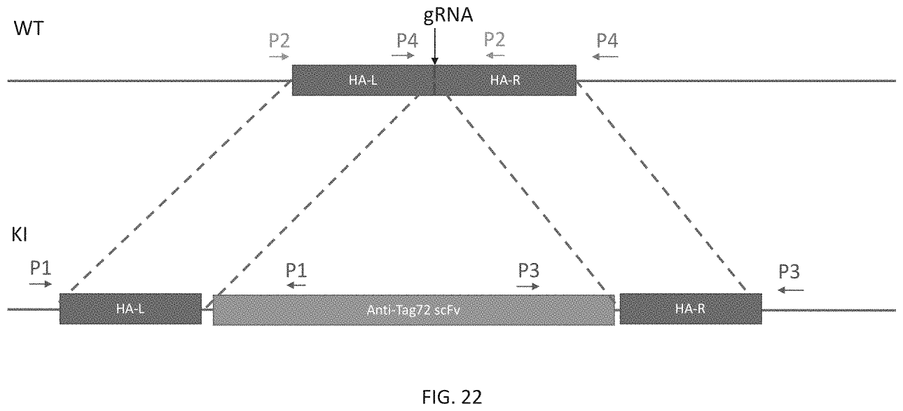

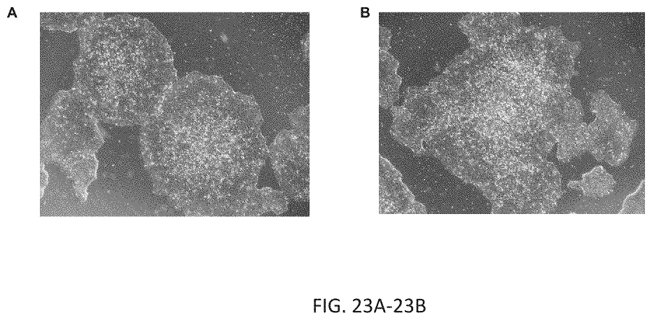

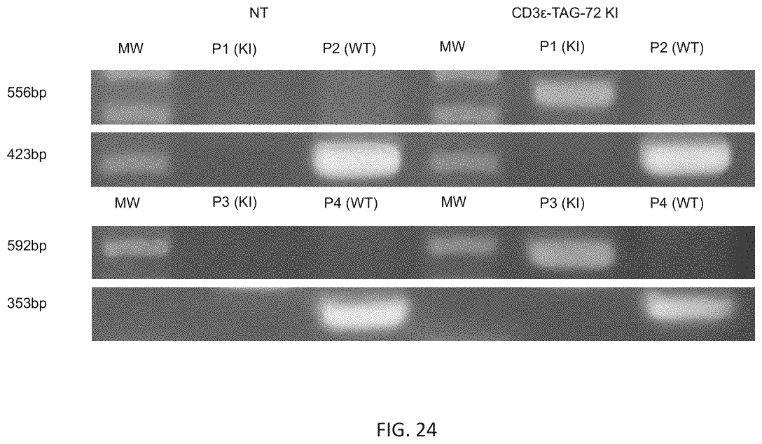

Stem cells such as induced pluripotent stem cells (iPSCs) can unlimitedly self-renew and differentiate into various cell types including hematopoietic stem cells (HSCs) and immune cells. Immune cells like T cells and NK cells had already been generated from iPSCs for cancer therapy (Themeli et al., Nat Biotechnol (2013) 31(10): p. 928-33; Li et al., Cell Stem Cell (2018) 23(2): p. 181-192 e185). FP T or FP NK cells can be derived from iPSCs, following similar methods (FIG. 21). To produce the anti-TAG-72/CD3c CRISPR FP T cells from iPSCs, CD3ϵ, gRNA-I RNP and anti-TAG-72 scFv donor DNA were transfected into iPSCs (H×2 iPS cell line) using Nucleofection according to the method as described in Example 1, and viable iPSC colonies with pluripotent stem cell like morphology were maintained for further clonal isolation and differentiation (FIG. 23). As the CD3ϵ receptor is not expressed at the iPSC stage, in order to verify the genotype of transfected iPSCs, genotyping PCR primers were designed flanking the two homological arms of the knock-in or wildtype allele in CD3ϵ locus (FIG. 22). PCR products of these flanking primers were visualized after 2% agarose gel electrophoresis, and PCR bands of knock-in allele, P1 and P3 were only detected in the transfected iPSCs (FIG. 24). These data indicate that the anti-TAG-72 say donor DNA was precisely incorporated into the CDR locus of iPSCs as the CD3ϵ CRISPR anti-TAG-72 FP T cells. After clonal selection, single clonal iPSCs can differentiate into HSCs and then to immune cells such as NK, NKT or T cells using known methods to provide the anti-TAG-72/CD3ϵ CRISPR FP immune cells for cancer therapy.

1-118. (Canceled)

119. A modified immune cell that recognizes a target antigen, comprising in its genome:
a nucleic acid sequence encoding an antigen recognition moiety derived from an antibody molecule for the target antigen inserted in an endogenous cell surface receptor gene to form a modified cell surface receptor gene,
wherein the insertion is at a specific site in the coding region of the endogenous cell surface receptor gene such that the endogenous cell surface receptor is modified to include the antigen recognition moiety in the extracellular domain, and
wherein expression of the modified cell surface receptor gene is under control of the endogenous cis-regulatory element at the endogenous cell surface receptor gene locus.

120. The modified immune cell of claim 119, wherein the modified immune cell comprises two or more modified cell surface receptor genes and recognizes two or more different target antigens.

121. The modified immune cell of claim 119, wherein the immune cell is selected from a T cell, a NKT cell, a NK cell or a macrophage.

122. The modified immune cell of claim 119, wherein the cell surface receptor is selected from CD2, CD3, CD4, CD8, TCR, CD28, NKG2D, CD94, CD16, NKp46,NKp30, NKp44, 2B4, IL-2Rα, IL-15Rα, IL-21R, CD69, CD27, DNAM1, DAP10, DAP12, FcRγ, IL-12R, or IL-18R.

123. The modified immune cell of claim 119, wherein the cell surface receptor is selected from CD3ϵ, CD3γ or CD35.

124. The modified immune cell of claim 119, wherein the cell surface receptor is selected from TCRα, TCRβ, TCRγ or TCRδ.

125. The modified immune cell of claim 119, wherein the insertion is an in-frame insertion within the extracellular domain-coding sequence in the endogenous cell surface receptor gene.

126. The modified immune cell of claim 119, wherein the insertion is an in-frame insertion immediately following or followed by the codon encoding the free end amino acid of the extracellular domain of the endogenous cell surface receptor.

127. The modified immune cell of claim 119, wherein the target antigen is a cell surface protein.

128. The modified immune cell of claim 119, wherein the target antigen is a tumor associated antigen.

129. The modified immune cell of claim 119, wherein the target antigen is selected from TAG-72, CD19, CD20, CD47, folate receptor alpha (FRa), or BCMA.

130. The modified immune cell of claim 119, wherein the target antigen is a viral protein, or a cell surface receptor or co-receptor that serves as the site for viral binding to a cell.

131. The modified immune cell of claim 130, wherein the viral protein is a viral protein expressed on the surface of virally-infected cells.

132. The modified immune cell of claim 119, wherein the antigen recognition moiety for the targeted antigen is a variable domain from a protein of the immunoglobulin superfamily.

133. The modified immune cell of claim 119, wherein the antigen recognition moiety for the targeted antigen is a scFv.

134. The modified immune cell of claim 119, wherein the nucleic acid sequence inserted encodes the antigen recognition moiety and a linker.

135. The modified immune cell of claim 119, wherein the size of the nucleic acid sequence is less than 1.5 kb.

136. The modified immune cell of claim 119, wherein the modified immune cell is differentiated from a modified stem cell comprising the modified cell surface receptor gene.


SEQUENCE LISTING (111 sequences)

SEQ ID NO: 1 - DNA, 20 bp, Artificial Sequence
  Synthetic oligonucleotide
gttggcgtttgggggcaaga

SEQ ID NO: 2 - DNA, 20 bp, Artificial Sequence
  Synthetic oligonucleotide
attttctagttggcgtttgg

SEQ ID NO: 3 - DNA, 20 bp, Artificial Sequence
  Synthetic oligonucleotide
cctctataggtatcttgaag

SEQ ID NO: 4 - DNA, 20 bp, Artificial Sequence
  Synthetic oligonucleotide
tctcctacctttgattgact

SEQ ID NO: 5 - DNA, 20 bp, Artificial Sequence
  Synthetic oligonucleotide
actttggcccagtcaatcaa

SEQ ID NO: 6 - DNA, 20 bp, Artificial Sequence
  Synthetic oligonucleotide
tggcccagtcaatcaaaggt

SEQ ID NO: 7 - DNA, 20 bp, Artificial Sequence
  Synthetic oligonucleotide
ggggccactagggacaggat

SEQ ID NO: 8 - DNA, 1425 bp, Artificial Sequence
  Synthetic or recombinant polynucleotide
tggtcctaactttttctgactcctgcttgtcaaaaattgtgggctcatagttaatgctagatgcttccttcctctatttccccccaaatttcctgggaacccctggtcaataccagcagtaagttccactgttctagggtgtagaaatggctgtgacccagcagcaagagggaaggacatcagatgtcatcagtggtcatactgcaacacagccctttttctgtttaggaatgcaggtacccacaacatttactaacactttttttttcttatttattttctagttggcgtttgggggcagcaggtgcagctccagcagtctgatgccgagcttgtgaaacctggcgcctccgtgaagatcagctgtaaagccagcggctacaccttcaccgaccacgccattcactgggtcaagcagaaccctgagcagggcctcgaatggatcggctacttcagccccggcaacgacgacttcaagtacaacgagcggttcaagggcaaagccacactgaccgccgataagagcagcagcacagcctacctgcagctgaacagcctgaccagcgaagatagcgccgtgtacttctgcacacggtccctgaacatggcctattggggccagggaaccagcgtgacagtttcttctggtggcggaggatctggcggaggtggaagcggcggaggcggatctgatatcgtgatgacacagagcccaagcagcctgcctgtgtccgtgggagagaaagtgaccctgagctgcaagagcagccagagcctgctgtactccggcaatcagaagaactacctggcctggtatcagcagaagcccggacagtctcccaagctgctgatctactgggccagcaccagagaaagcggcgtgcccgatagattcacaggcagcggcagcggaaccgacttcacactgtctatcagcagcgtggaaaccgaggacctggccgtgtattactgccagcagtactacagctaccctctgacctttggagccggcaccaagctggtgcttaaaagagcggccgcaatcgaagtgatgtaccctcctccataccttggtggtggtggcagtggtggcggtggaagcggaggcggaggttctctcgagcaagatggtgagatatgctttctttctttcttttttatgaaatcaccccatcattctttgtagttatgaatggagctttctcttaggcctcccacagaacttccacagaggtcaggaaaaggagtttctgccatctacccctttgactttcctcacaagtctggagatatttctagcccagaagagggaagcaacagaggcaggaaataatgagtcttaaccatacaaaagaaaaattgagacttaaatgaagttgaaagcactaacagttttcatttgtttgcatttcatatttgatgtga

SEQ ID NO: 9 - DNA, 1440 bp, Artificial Sequence
  Synthetic or recombinant polynucleotide
tggtcctaactttttctgactcctgcttgtcaaaaattgtgggctcatagttaatgctagatgcttccttcctctatttccccccaaatttcctgggaacccctggtcaataccagcagtaagttccactgttctagggtgtagaaatggctgtgacccagcagcaagagggaaggacatcagatgtcatcagtggtcatactgcaacacagccctttttctgtttaggaatgcaggtacccacaacatttactaacactttttttttcttatttattttctagttggcgtttgggggcagcaggtgcagctccagcagtctgatgccgagcttgtgaaacctggcgcctccgtgaagatcagctgtaaagccagcggctacaccttcaccgaccacgccattcactgggtcaagcagaaccctgagcagggcctcgaatggatcggctacttcagccccggcaacgacgacttcaagtacaacgagcggttcaagggcaaagccacactgaccgccgataagagcagcagcacagcctacctgcagctgaacagcctgaccagcgaagatagcgccgtgtacttctgcacacggtccctgaacatggcctattggggccagggaaccagcgtgacagtttcttctggtggcggaggatctggcggaggtggaagcggcggaggcggatctgatatcgtgatgacacagagcccaagcagcctgcctgtgtccgtgggagagaaagtgaccctgagctgcaagagcagccagagcctgctgtactccggcaatcagaagaactacctggcctggtatcagcagaagcccggacagtctcccaagctgctgatctactgggccagcaccagagaaagcggcgtgcccgatagattcacaggcagcggcagcggaaccgacttcacactgtctatcagcagcgtggaaaccgaggacctggccgtgtattactgccagcagtactacagctaccctctgacctttggagccggcaccaagctggtgcttaaaagagactacaaagacgatgacgacaagatcgaagtgatgtaccctcctccataccttggtggtggtggcagtggtggcggtggaagcggaggcggaggttctctcgagcaagatggtgagatatgctttctttctttcttttttatgaaatcaccccatcattctttgtagttatgaatggagctttctcttaggcctcccacagaacttccacagaggtcaggaaaaggagtttctgccatctacccctttgactttcctcacaagtctggagatatttctagcccagaagagggaagcaacagaggcaggaaataatgagtcttaaccatacaaaagaaaaattgagacttaaatgaagttgaaagcactaacagttttcatttgtttgcatttcatatttgatgtga

SEQ ID NO: 10 - DNA, 1419 bp, Artificial Sequence
  Synthetic or recombinant polynucleotide
tggtcctaactttttctgactcctgcttgtcaaaaattgtgggctcatagttaatgctagatgcttccttcctctatttccccccaaatttcctgggaacccctggtcaataccagcagtaagttccactgttctagggtgtagaaatggctgtgacccagcagcaagagggaaggacatcagatgtcatcagtggtcatactgcaacacagccctttttctgtttaggaatgcaggtacccacaacatttactaacactttttttttcttatttattttctagttggcgtttgggggcaggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcagcggccgcaatcgaagtgatgtaccctcctccataccttggtggtggtggcagtggtggcggtggaagcggaggcggaggttctctcgagcaagatggtgagatatgctttctttctttcttttttatgaaatcaccccatcattctttgtagttatgaatggagctttctcttaggcctcccacagaacttccacagaggtcaggaaaaggagtttctgccatctacccctttgactttcctcacaagtctggagatatttctagcccagaagagggaagcaacagaggcaggaaataatgagtcttaaccatacaaaagaaaaattgagacttaaatgaagttgaaagcactaacagttttcatttgtttgcatttcatatttgatgtga

SEQ ID NO: 11 - DNA, 1479 bp, Artificial Sequence
  Synthetic or recombinant polynucleotide
tactcctcatcagtggtctggattcatttccaggaaggtgcttgggtcaatcctgctctgaggggtcctcttttgcaccaaggccccaagacctgtcctctgcttgaatggcatcctcatgtcccccttgacccctgcagttggtggggaactttctgaaggctttttcttgtgacttctgtctctcttgatggaagccataaactgtcctggaccaggaaaatggtctctgttaagtatgagcttccgcagaacaaagggcttggtgcagatcaaagagctgtctccaactgtgatattttttcccctttagtgagcccccaggtgcagctccagcagtctgatgccgagcttgtgaaacctggcgcctccgtgaagatcagctgtaaagccagcggctacaccttcaccgaccacgccattcactgggtcaagcagaaccctgagcagggcctcgaatggatcggctacttcagccccggcaacgacgacttcaagtacaacgagcggttcaagggcaaagccacactgaccgccgataagagcagcagcacagcctacctgcagctgaacagcctgaccagcgaagatagcgccgtgtacttctgcacacggtccctgaacatggcctattggggccagggaaccagcgtgacagtttcttctggtggcggaggatctggcggaggtggaagcggcggaggcggatctgatatcgtgatgacacagagcccaagcagcctgcctgtgtccgtgggagagaaagtgaccctgagctgcaagagcagccagagcctgctgtactccggcaatcagaagaactacctggcctggtatcagcagaagcccggacagtctcccaagctgctgatctactgggccagcaccagagaaagcggcgtgcccgatagattcacaggcagcggcagcggaaccgacttcacactgtctatcagcagcgtggaaaccgaggacctggccgtgtattactgccagcagtactacagctaccctctgacctttggagccggcaccaagctggtgcttaaaagagactacaaagacgatgacgacaagatcgaagtgatgtaccctcctccataccttggtggtggtggcagtggtggcggtggaagcggaggcggaggttctctcgagtttaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaagtacgtgcttcctgaaccctttgggttggaatggatagggcttctggatgtgagaactttctggctagagagggatatggtgaacccatctgttctctaaacaaagtgag

SEQ ID NO: 12 - DNA, 1480 bp, Artificial Sequence
  Synthetic or recombinant polynucleotide
catgcataaactcaggtatggtctaaggatccaccacagataacaaagccactcctgtcactcatgaaattccaacggttagaaacaccctcccaggataccaggacaaagatgagacaaattgtttattatcaccttgaattcaattgagtgatttagtctacaatccggaaaactaagtatagatactaccattttcatggatttggatctttcttcatcttggcctcaaataaccatggaaatacttcagggcatctgaacaactccatgcccagctaatactctatctctcttctgtctttacaggtactttggcccaggtgcagctccagcagtctgatgccgagcttgtgaaacctggcgcctccgtgaagatcagctgtaaagccagcggctacaccttcaccgaccacgccattcactgggtcaagcagaaccctgagcagggcctcgaatggatcggctacttcagccccggcaacgacgacttcaagtacaacgagcggttcaagggcaaagccacactgaccgccgataagagcagcagcacagcctacctgcagctgaacagcctgaccagcgaagatagcgccgtgtacttctgcacacggtccctgaacatggcctattggggccagggaaccagcgtgacagtttcttctggtggcggaggatctggcggaggtggaagcggcggaggcggatctgatatcgtgatgacacagagcccaagcagcctgcctgtgtccgtgggagagaaagtgaccctgagctgcaagagcagccagagcctgctgtactccggcaatcagaagaactacctggcctggtatcagcagaagcccggacagtctcccaagctgctgatctactgggccagcaccagagaaagcggcgtgcccgatagattcacaggcagcggcagcggaaccgacttcacactgtctatcagcagcgtggaaaccgaggacctggccgtgtattactgccagcagtactacagctaccctctgacctttggagccggcaccaagctggtgcttaaaagagactacaaagacgatgacgacaagatcgaagtgatgtaccctcctccataccttggtggtggtggcagtggtggcggtggaagcggaggcggaggttctctcgagcagtcaatcaaaggtaggagaaatggcttctttctatactcagactcagaatattgacggaaatttggcttcctacaacagtagtcctacaggagcgaacagtttagaatgaatgaaatgacggggatagagaggtgatgtctctattgtcaaccaaatcagtgacctgacataacctgttccgggcagcttgcctgtagctaagcatttaactggtctcttacaggagaagcaggaccctagtagctagggacacatctcaaactgtgacccatgaaccagtaggtattggcgtcacctgggatcctgacgttagtctctgt

SEQ ID NO: 13 - DNA, 2786 bp, Artificial Sequence
  Synthetic or recombinant polynucleotide
gtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctaggtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaagattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctc

SEQ ID NO: 14 - DNA, 2386 bp, Artificial Sequence
  Synthetic or recombinant polynucleotide
atcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctaggtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaagattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttct

SEQ ID NO: 15 - DNA, 1985 bp, Artificial Sequence
  Synthetic or recombinant polynucleotide
caggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctaggtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaagattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcag

SEQ ID NO: 16 - DNA, 1786 bp, Artificial Sequence
  Synthetic or recombinant polynucleotide
gagctgggaccaccttatattcccagggccggttaatgtggctctggttctgggtacttttatctgtcccctccaccccacagtggggccactagggacagactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctaggtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaagattggtgacagaaaagccccatccttaggcctcctccttcctagtctcctgatattgggtctaacccccacctcctgttaggcagattccttatctggtg

SEQ ID NO: 17 - DNA, 3018 bp, Artificial Sequence
  Synthetic or recombinant polynucleotide
atcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagactagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgactagctagtagctaggtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaagattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttct

SEQ ID NO: 18 - DNA, 3936 bp, Artificial Sequence
  Synthetic or recombinant polynucleotide
gctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggagttcggcctgcgctgggtgttcctggtggccatcctgaaggacgtgcagtgccaggtgcagctgcagcagagcgacgccgagctggtgaagcccggcgccagcgtgaagatcagctgcaaggccagcggctacaccttcaccgaccacgccatccactgggtgaagcagaaccccgagcagggcctggagtggatcggctacttcagccccggcaacgacgacttcaagtacaacgagcgcttcaagggcaaggccaccctgaccgccgacaagagcagcagcaccgcctacctgcagctgaacagcctgaccagcgaggacagcgccgtgtacttctgcacccgcagcctgaacatggcctactggggccagggcaccagcgtgaccgtgagcagcggcggcggcggcagcggcggcggcggcagcggcggcggcggcagcgacatcgtgatgacccagagccccagcagcctgcccgtgagcgtgggcgagaaggtgaccctgagctgcaagagcagccagagcctgctgtacagcggcaaccagaagaactacctggcctggtaccagcagaagcccggccagagccccaagctcctgatctactgggccagcacccgcgagagcggcgtgcccgaccgcttcaccggcagcggcagcggcaccgacttcaccctgagcatcagcagcgtggagaccgaggacctggccgtgtactactgccagcagtactacagctaccccctgaccttcggcgccggcaccaagctggtgctgaagcgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaagattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 19 - DNA, 3097 bp, Artificial Sequence
  Synthetic or recombinant polynucleotide
atcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggagttcggcctgcgctgggtgttcctggtggccatcctgaaggacgtgcagtgccaggtgcagctgcagcagagcgacgccgagctggtgaagcccggcgccagcgtgaagatcagctgcaaggccagcggctacaccttcaccgaccacgccatccactgggtgaagcagaaccccgagcagggcctggagtggatcggctacttcagccccggcaacgacgacttcaagtacaacgagcgcttcaagggcaaggccaccctgaccgccgacaagagcagcagcaccgcctacctgcagctgaacagcctgaccagcgaggacagcgccgtgtacttctgcacccgcagcctgaacatggcctactggggccagggcaccagcgtgaccgtgagcagcggcggcggcggcagcggcggcggcggcagcggcggcggcggcagcgacatcgtgatgacccagagccccagcagcctgcccgtgagcgtgggcgagaaggtgaccctgagctgcaagagcagccagagcctgctgtacagcggcaaccagaagaactacctggcctggtaccagcagaagcccggccagagccccaagctcctgatctactgggccagcacccgcgagagcggcgtgcccgaccgcttcaccggcagcggcagcggcaccgacttcaccctgagcatcagcagcgtggagaccgaggacctggccgtgtactactgccagcagtactacagctaccccctgaccttcggcgccggcaccaagctggtgctgaagcgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaagattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttct

SEQ ID NO: 20 - DNA, 3527 bp, Artificial Sequence
  Synthetic or recombinant polynucleotide
tgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtacacggcagggtcagggttctggatatcactagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattcgccaccatggagttcggcctgcgctgggtgttcctggtggccatcctgaaggacgtgcagtgccaggtgcagctgcagcagagcgacgccgagctggtgaagcccggcgccagcgtgaagatcagctgcaaggccagcggctacaccttcaccgaccacgccatccactgggtgaagcagaaccccgagcagggcctggagtggatcggctacttcagccccggcaacgacgacttcaagtacaacgagcgcttcaagggcaaggccaccctgaccgccgacaagagcagcagcaccgcctacctgcagctgaacagcctgaccagcgaggacagcgccgtgtacttctgcacccgcagcctgaacatggcctactggggccagggcaccagcgtgaccgtgagcagcggcggcggcggcagcggcggcggcggcagcggcggcggcggcagcgacatcgtgatgacccagagccccagcagcctgcccgtgagcgtgggcgagaaggtgaccctgagctgcaagagcagccagagcctgctgtacagcggcaaccagaagaactacctggcctggtaccagcagaagcccggccagagccccaagctcctgatctactgggccagcacccgcgagagcggcgtgcccgaccgcttcaccggcagcggcagcggcaccgacttcaccctgagcatcagcagcgtggagaccgaggacctggccgtgtactactgccagcagtactacagctaccccctgaccttcggcgccggcaccaagctggtgctgaagcgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaatgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaacctgaaatgcagggctacttaataatact

SEQ ID NO: 21 - DNA, 2895 bp, Artificial Sequence
  Synthetic or recombinant polynucleotide
tgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtacacggcagggtcagggttctggatatcactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggagttcggcctgcgctgggtgttcctggtggccatcctgaaggacgtgcagtgccaggtgcagctgcagcagagcgacgccgagctggtgaagcccggcgccagcgtgaagatcagctgcaaggccagcggctacaccttcaccgaccacgccatccactgggtgaagcagaaccccgagcagggcctggagtggatcggctacttcagccccggcaacgacgacttcaagtacaacgagcgcttcaagggcaaggccaccctgaccgccgacaagagcagcagcaccgcctacctgcagctgaacagcctgaccagcgaggacagcgccgtgtacttctgcacccgcagcctgaacatggcctactggggccagggcaccagcgtgaccgtgagcagcggcggcggcggcagcggcggcggcggcagcggcggcggcggcagcgacatcgtgatgacccagagccccagcagcctgcccgtgagcgtgggcgagaaggtgaccctgagctgcaagagcagccagagcctgctgtacagcggcaaccagaagaactacctggcctggtaccagcagaagcccggccagagccccaagctcctgatctactgggccagcacccgcgagagcggcgtgcccgaccgcttcaccggcagcggcagcggcaccgacttcaccctgagcatcagcagcgtggagaccgaggacctggccgtgtactactgccagcagtactacagctaccccctgaccttcggcgccggcaccaagctggtgctgaagcgcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgagtcgacgcggccgcgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttcgaatgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaacctgaaatgcagggctacttaataatact

SEQ ID NO: 22 - protein, 482 aa, Artificial Sequence
  recombinant polypeptide (the sequences are all one letter code for proteins)
MQSGTHWRVLGLCLLSVGVWGQQVQLQQSDAELVKPGASVKISCKASGYTFTDHAIHWVKQNPEQGLEWIGYFSPGNDDFKYNERFKGKATLTADKSSSTAYLQLNSLTSEDSAVYFCTRSLNMAYWGQGTSVTVSSGGGGSGGGGSGGGGSDIVMTQSPSSLPVSVGEKVTLSCKSSQSLLYSGNQKNYLAWYQQKPGQSPKLLIYWASTRESGVPDRFTGSGSGTDFTLSISSVETEDLAVYYCQQYYSYPLTFGAGTKLVLKRAAAIEVMYPPPYLGGGGSGGGGSGGGGSLEQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 23 - protein, 480 aa, Artificial Sequence
  recombinant polypeptide (the sequences are all one letter code for proteins)
MQSGTHWRVLGLCLLSVGVWGQDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLGGGGSGGGGSGGGGSLEQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 24 - protein, 445 aa, Artificial Sequence
  recombinant polypeptide (the sequences are all one letter code for proteins)
MEHSTFLSGLVLATLLSQVSPQVQLQQSDAELVKPGASVKISCKASGYTFTDHAIHWVKQNPEQGLEWIGYFSPGNDDFKYNERFKGKATLTADKSSSTAYLQLNSLTSEDSAVYFCTRSLNMAYWGQGTSVTVSSGGGGSGGGGSGGGGSDIVMTQSPSSLPVSVGEKVTLSCKSSQSLLYSGNQKNYLAWYQQKPGQSPKLLIYWASTRESGVPDRFTGSGSGTDFTLSISSVETEDLAVYYCQQYYSYPLTFGAGTKLVLKRAAAIEVMYPPPYLGGGGSGGGGSGGGGSLEFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 25 - protein, 456 aa, Artificial Sequence
  recombinant polypeptide (the sequences are all one letter code for proteins)
MEQGKGLAVLILAIILLQGTLAQVQLQQSDAELVKPGASVKISCKASGYTFTDHAIHWVKQNPEQGLEWIGYFSPGNDDFKYNERFKGKATLTADKSSSTAYLQLNSLTSEDSAVYFCTRSLNMAYWGQGTSVTVSSGGGGSGGGGSGGGGSDIVMTQSPSSLPVSVGEKVTLSCKSSQSLLYSGNQKNYLAWYQQKPGQSPKLLIYWASTRESGVPDRFTGSGSGTDFTLSISSVETEDLAVYYCQQYYSYPLTFGAGTKLVLKRAAAIEVMYPPPYLGGGGSGGGGSGGGGSLEQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 26 - DNA, 20 bp, Artificial Sequence
  Synthetic oligonucleotide
tcctctataggtatcttgaa

SEQ ID NO: 27 - DNA, 20 bp, Artificial Sequence
  Synthetic oligonucleotide
ttcctctataggtatcttga

SEQ ID NO: 28 - DNA, 20 bp, Artificial Sequence
  Synthetic oligonucleotide
agctttatctcttggcacag

SEQ ID NO: 29 - DNA, 20 bp, Artificial Sequence
  Synthetic oligonucleotide
ggcacagaggcacagagctt

SEQ ID NO: 30 - DNA, 20 bp, Artificial Sequence
  Synthetic oligonucleotide
ctctcagctggtacacggca

SEQ ID NO: 31 - DNA, 20 bp, Artificial Sequence
  Synthetic oligonucleotide
agagtctctcagctggtaca

SEQ ID NO: 32 - DNA, 20 bp, Artificial Sequence
  Synthetic oligonucleotide
gctggtacacggcagggtca

SEQ ID NO: 33 - DNA, 20 bp, Artificial Sequence
  Synthetic oligonucleotide
aggtcgctgtgtttgagcca

SEQ ID NO: 34 - DNA, 20 bp, Artificial Sequence
  Synthetic oligonucleotide
aggtcgctgtgtttgagcca

SEQ ID NO: 35 - DNA, 20 bp, Artificial Sequence
  Synthetic oligonucleotide
catcaagttgtttatctatg

SEQ ID NO: 36 - DNA, 20 bp, Artificial Sequence
  Synthetic oligonucleotide
gcatcaagttgtttatctat

SEQ ID NO: 37 - DNA, 20 bp, Artificial Sequence
  Synthetic oligonucleotide
tgcatcaagttgtttatcta

SEQ ID NO: 38 - DNA, 20 bp, Artificial Sequence
  Synthetic oligonucleotide
aataagttgattatatttgc

SEQ ID NO: 39 - DNA, 20 bp, Artificial Sequence
  Synthetic oligonucleotide
aaaacggatggtttggtatg

SEQ ID NO: 40 - DNA, 20 bp, Artificial Sequence
  Synthetic oligonucleotide
accaaaatcttgtttcctgg

SEQ ID NO: 41 - DNA, 20 bp, Artificial Sequence
  Synthetic oligonucleotide
caccaaaatcttgtttcctg

SEQ ID NO: 42 - DNA, 20 bp, Artificial Sequence
  Synthetic oligonucleotide
ctcagggaaagaaagtggtg

SEQ ID NO: 43 - DNA, 20 bp, Artificial Sequence
  Synthetic oligonucleotide
cagggaaagaaagtggtgct

SEQ ID NO: 44 - DNA, 20 bp, Artificial Sequence
  Synthetic oligonucleotide
cagggaaagaaagtggtgct

SEQ ID NO: 45 - DNA, 20 bp, Artificial Sequence
  Synthetic oligonucleotide
ccggaactggctcggcctgg

SEQ ID NO: 46 - DNA, 20 bp, Artificial Sequence
  Synthetic oligonucleotide
cacccggaactggctcggcc

SEQ ID NO: 47 - DNA, 20 bp, Artificial Sequence
  Synthetic oligonucleotide
agccagttccgggtgtcgcc

SEQ ID NO: 48 - DNA, 20 bp, Artificial Sequence
  Synthetic oligonucleotide
ggcaggtaagggcctgtggg

SEQ ID NO: 49 - DNA, 20 bp, Artificial Sequence
  Synthetic oligonucleotide
cctggctgccaggcaggtaa

SEQ ID NO: 50 - DNA, 20 bp, Artificial Sequence
  Synthetic oligonucleotide
gccgccggcgacgcggggta

SEQ ID NO: 51 - DNA, 20 bp, Artificial Sequence
  Synthetic oligonucleotide
ctccggccgccggcgacgcg

SEQ ID NO: 52 - DNA, 20 bp, Artificial Sequence
  Synthetic oligonucleotide
cggtgtagcagacgaggtcg

SEQ ID NO: 53 - DNA, 20 bp, Artificial Sequence
  Synthetic oligonucleotide
aggtcggggcagccccagcc

SEQ ID NO: 54 - DNA, 20 bp, Artificial Sequence
  Synthetic oligonucleotide
aaggaaggactcacgctgct

SEQ ID NO: 55 - DNA, 20 bp, Artificial Sequence
  Synthetic oligonucleotide
gaaggaaggactcacgctgc

SEQ ID NO: 56 - DNA, 20 bp, Artificial Sequence
  Synthetic oligonucleotide
aggctctcaggcacaatcca

SEQ ID NO: 57 - DNA, 20 bp, Artificial Sequence
  Synthetic oligonucleotide
tcaggcacaatccaaggctc

SEQ ID NO: 58 - DNA, 20 bp, Artificial Sequence
  Synthetic oligonucleotide
ctgggacacccagagagcac

SEQ ID NO: 59 - DNA, 20 bp, Artificial Sequence
  Synthetic oligonucleotide
cccaggatcctgtgctctct

SEQ ID NO: 60 - DNA, 20 bp, Artificial Sequence
  Synthetic oligonucleotide
atacgtacatctgcatgcaa

SEQ ID NO: 61 - DNA, 20 bp, Artificial Sequence
  Synthetic oligonucleotide
gttcctggcttttattgaga

SEQ ID NO: 62 - DNA, 20 bp, Artificial Sequence
  Synthetic oligonucleotide
gaccaaaccgactagacagg

SEQ ID NO: 63 - DNA, 20 bp, Artificial Sequence
  Synthetic oligonucleotide
agggaccaaaccgactagac

SEQ ID NO: 64 - DNA, 20 bp, Artificial Sequence
  Synthetic oligonucleotide
tttacttcctcctgtctagt

SEQ ID NO: 65 - DNA, 20 bp, Artificial Sequence
  Synthetic oligonucleotide
ttctctttcctaggatgcca

SEQ ID NO: 66 - DNA, 20 bp, Artificial Sequence
  Synthetic oligonucleotide
gttctctttcctaggatgcc

SEQ ID NO: 67 - DNA, 20 bp, Artificial Sequence
  Synthetic oligonucleotide
cagctgatccctggcatcct

SEQ ID NO: 68 - DNA, 20 bp, Artificial Sequence
  Synthetic oligonucleotide
gctcatttaaatgtttcttg

SEQ ID NO: 69 - DNA, 20 bp, Artificial Sequence
  Synthetic oligonucleotide
gtgaagataaaaatcgttat

SEQ ID NO: 70 - DNA, 20 bp, Artificial Sequence
  Synthetic oligonucleotide
caaggcattcgtaatctctt

SEQ ID NO: 71 - DNA, 20 bp, Artificial Sequence
  Synthetic oligonucleotide
caaagagattacgaatgcct

SEQ ID NO: 72 - DNA, 20 bp, Artificial Sequence
  Synthetic oligonucleotide
atgtttccttattatttgta

SEQ ID NO: 73 - DNA, 20 bp, Artificial Sequence
  Synthetic oligonucleotide
aacaaaccttacaaataata

SEQ ID NO: 74 - DNA, 20 bp, Artificial Sequence
  Synthetic oligonucleotide
aatgtgagaagaatttatac

SEQ ID NO: 75 - DNA, 20 bp, Artificial Sequence
  Synthetic oligonucleotide
agtagctgagagccccacca

SEQ ID NO: 76 - DNA, 20 bp, Artificial Sequence
  Synthetic oligonucleotide
cctctctgggcagctcttgg

SEQ ID NO: 77 - DNA, 20 bp, Artificial Sequence
  Synthetic oligonucleotide
tctgggcagctcttgggggc

SEQ ID NO: 78 - DNA, 20 bp, Artificial Sequence
  Synthetic oligonucleotide
tctgggcagctcttgggggc

SEQ ID NO: 79 - DNA, 20 bp, Artificial Sequence
  Synthetic oligonucleotide
tatttcagctctatgtgaag

SEQ ID NO: 80 - DNA, 20 bp, Artificial Sequence
  Synthetic oligonucleotide
ctctatgtgaagaggtgctt

SEQ ID NO: 81 - DNA, 20 bp, Artificial Sequence
  Synthetic oligonucleotide
gctgcagctcagacgactcc

SEQ ID NO: 82 - DNA, 20 bp, Artificial Sequence
  Synthetic oligonucleotide
cgtctgagctgcagccactg

SEQ ID NO: 83 - DNA, 20 bp, Artificial Sequence
  Synthetic oligonucleotide
gacaggacggagacctgagg

SEQ ID NO: 84 - DNA, 20 bp, Artificial Sequence
  Synthetic oligonucleotide
ctggacaggacggagacctg

SEQ ID NO: 85 - DNA, 20 bp, Artificial Sequence
  Synthetic oligonucleotide
gggcctgggcctggacagga

SEQ ID NO: 86 - DNA, 20 bp, Artificial Sequence
  Synthetic oligonucleotide
gcagagctgaggctctccca

SEQ ID NO: 87 - DNA, 20 bp, Artificial Sequence
  Synthetic oligonucleotide
tctatcccctcagcggccct

SEQ ID NO: 88 - DNA, 20 bp, Artificial Sequence
  Synthetic oligonucleotide
acagcactcactggttctgc

SEQ ID NO: 89 - DNA, 20 bp, Artificial Sequence
  Synthetic oligonucleotide
ggttctgcaggcagctgcaa

SEQ ID NO: 90 - DNA, 20 bp, Artificial Sequence
  Synthetic oligonucleotide
gtcctgaaaacagcactcac

SEQ ID NO: 91 - DNA, 20 bp, Artificial Sequence
  Synthetic oligonucleotide
ctgttgattaaagcaaaaat

SEQ ID NO: 92 - DNA, 20 bp, Artificial Sequence
  Synthetic oligonucleotide
attgcagatgcgtgcaagag

SEQ ID NO: 93 - DNA, 20 bp, Artificial Sequence
  Synthetic oligonucleotide
ccttcaaccacagtaatgtg

SEQ ID NO: 94 - DNA, 20 bp, Artificial Sequence
  Synthetic oligonucleotide
cttatatctgtaagcactgc

SEQ ID NO: 95 - DNA, 1434 bp, Artificial Sequence
  Anti-TAG-72 scFv donor DNA with Flag tag
aactcatcataccttttaacttaaatagttaaaatgtataaagtgtgaaacctgcctgtcacatagtaagtactcaagattttagctattggtctatgggatccctcagactagggacacagagcttggggacaaggggaggcagatggaggggatatgctttaaggtgctcctaggttctgggctcctcatactcatccccacccaattggcttaagggaagtcacatccagctgcccacattgctcaggcctgacactcttcccgctactttcagcttgggttctctttcctaggatgccaggtgcagctccagcagtctgatgccgagcttgtgaaacctggcgcctccgtgaagatcagctgtaaagccagcggctacaccttcaccgaccacgccattcactgggtcaagcagaaccctgagcagggcctcgaatggatcggctacttcagccccggcaacgacgacttcaagtacaacgagcggttcaagggcaaagccacactgaccgccgataagagcagcagcacagcctacctgcagctgaacagcctgaccagcgaagatagcgccgtgtacttctgcacacggtccctgaacatggcctattggggccagggaaccagcgtgacagtttcttctggtggcggaggatctggcggaggtggaagcggcggaggcggatctgatatcgtgatgacacagagcccaagcagcctgcctgtgtccgtgggagagaaagtgaccctgagctgcaagagcagccagagcctgctgtactccggcaatcagaagaactacctggcctggtatcagcagaagcccggacagtctcccaagctgctgatctactgggccagcaccagagaaagcggcgtgcccgatagattcacaggcagcggcagcggaaccgacttcacactgtctatcagcagcgtggaaaccgaggacctggccgtgtattactgccagcagtactacagctaccctctgacctttggagccggcaccaagctggtgcttaaaagagactacaaagacgatgacgacaagatcgaagtgatgtaccctcctccataccttggtggtggtggcagtggtggcggtggaagcggaggcggaggttcttgccagggatcagctgaccatgtggttagcatctcgggagtgcctcttcagttacaaccaaacagcatacagacgaaggttgacagcattgcatggaagaagttgctgccctcacaaaatggatttcatcacatattgaagtgggagaatggctctttgccttccaatacttccaatgatagattcagttttatagtcaagaacttgagtcttctcatcaaggcagctcagcagcaggacagtggcctctactgcctggaggtcaccagtatatctggaaaagttcagacagccacgttcca

SEQ ID NO: 96 - DNA, 1476 bp, Artificial Sequence
  CD28 anti-TAG-72 scFv donor DNA
cccaatgcatgattaatattaatgtctcaggaatatagatttaaaaattgtgagtgcctagctgtggtgctcaatgctaacagaaagccatgtactgccttctgggtgaaaacatgctccattgttccattcaaaagaggatgaaaataatattgaaatgttaacttttgaactctcagtataattatcccctgaagaggtcctacatcttctgccaagagaaaacctttgctggagatctgttcatttagttaattaatatattttgctctcaggaagaaaaattatccttatattcttgttctaagcaaatgatttttttttcccccaggacaagtgcagctccagcagtctgatgccgagcttgtgaaacctggcgcctccgtgaagatcagctgtaaagccagcggctacaccttcaccgaccacgccattcactgggtcaagcagaaccctgagcagggcctcgaatggatcggctacttcagccccggcaacgacgacttcaagtacaacgagcggttcaagggcaaagccacactgaccgccgataagagcagcagcacagcctacctgcagctgaacagcctgaccagcgaagatagcgccgtgtacttctgcacacggtccctgaacatggcctattggggccagggaaccagcgtgacagtttcttctggtggcggaggatctggcggaggtggaagcggcggaggcggatctgatatcgtgatgacacagagcccaagcagcctgcctgtgtccgtgggagagaaagtgaccctgagctgcaagagcagccagagcctgctgtactccggcaatcagaagaactacctggcctggtatcagcagaagcccggacagtctcccaagctgctgatctactgggccagcaccagagaaagcggcgtgcccgatagattcacaggcagcggcagcggaaccgacttcacactgtctatcagcagcgtggaaaccgaggacctggccgtgtattactgccagcagtactacagctaccctctgacctttggagccggcaccaagctggtgcttaaaagaatcgaagtgatgtaccctcctccataccttggtggtggtggcagtggtggcggtggaagcggaggcggaggttctctcgagggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaag

SEQ ID NO: 97 - DNA, 75 bp, Artificial Sequence
  a polynucleotide sequence within CD3 epsilon Exon 3 having indels
  misc_feature (26)..(27): N represents any nucleotide
agaaagaaagcatatctcaccatctnntgcccccaaacgccaactagaaaataaataagaaaaaaaaagtgttag

SEQ ID NO: 98 - DNA, 75 bp, Artificial Sequence
  a polynucleotide sequence within CD3 epsilon Exon 3 having indels
  misc_feature (26)..(26): N represents any nucleotide
agaaagaaagcatatctcaccatctntgcccccaaacgccaactagaaaataaataagaaaaaaaaagtgttagt

SEQ ID NO: 99 - DNA, 65 bp, Artificial Sequence
  a polynucleotide sequence within CD3 epsilon Exon 3 having indels
agaaagaaagcatatctcccccaaacgccaactagaaaataaataagaaaaaaaaagtgttagta

SEQ ID NO: 100 - DNA, 58 bp, Artificial Sequence
  a polynucleotide sequence within CD3 epsilon Exon 3 having indels
agaaagaaagcatatctacgccaactagaaaataaataagaaaaaaaaagtgttagta

SEQ ID NO: 101 - DNA, 68 bp, Artificial Sequence
  a polynucleotide sequence within CD3 epsilon Exon 3 having indels
agaaagaaagcatatctctgcccccaaacgccaactagaaaataaataagaaaaaaaaagtgttagta

SEQ ID NO: 102 - DNA, 73 bp, Artificial Sequence
  a polynucleotide sequence within CD3 epsilon Exon 3 having indels
agaaagaaagcatatctcaccattgcccccaaacgccaactagaaaataaataagaaaaaaaaagtgttagta

SEQ ID NO: 103 - DNA, 75 bp, Artificial Sequence
  a polynucleotide sequence within CD3 epsilon Exon 3
agaaagaaagcatatctcaccatcttgcccccaaacgccaactagaaaataaataagaaaaaaaaagtgttagta

SEQ ID NO: 104 - DNA, 20 bp, Artificial Sequence
  Synthetic Oligonucleotide
caatgttcaaaatggaggct

SEQ ID NO: 105 - DNA, 19 bp, Artificial Sequence
  Synthetic Oligonucleotide
gaaccgctcgttgtacttg

SEQ ID NO: 106 - DNA, 20 bp, Artificial Sequence
  Synthetic Oligonucleotide
caatgttcaaaatggaggct

SEQ ID NO: 107 - DNA, 20 bp, Artificial Sequence
  Synthetic Oligonucleotide
tacaaagaatgatggggtga

SEQ ID NO: 108 - DNA, 18 bp, Artificial Sequence
  Synthetic Oligonucleotide
cggcgtgcccgatagatt

SEQ ID NO: 109 - DNA, 18 bp, Artificial Sequence
  Synthetic Oligonucleotide
attctggctacgtctcct

SEQ ID NO: 110 - DNA, 20 bp, Artificial Sequence
  Synthetic Oligonucleotide
attttctagttggcgtttgg

SEQ ID NO: 111 - DNA, 18 bp, Artificial Sequence
  Synthetic Oligonucleotide
attctggctacgtctcct